(19) United States
(12) Patent Application Publication
(10) Pub. No.: US 2012/0190743 A1
(43) Pub. Date: Jul. 26, 2012

(54) COMPOUNDS FOR TREATING DISORDERS OR DISEASES ASSOCIATED WITH NEUROKININ 2 RECEPTOR ACTIVITY

(75) Inventors: Jerald Bain, Toronto (CA); Joel Sadavoy, Toronto (CA); Hao Chen, Columbia, MD (US); Xiaoyu Shen, Columbia, MD (US)

(73) Assignee: UNITED PARAGON ASSOCIATES INC., Guelph, ON (CA)

(21) Appl. No.: 13/394,067

(22) PCT Filed: Sep. 7, 2010
(86) PCT No.: PCT/US10/48006

§ 371 Date: Apr. 12, 2012

Related U.S. Application Data
(60) Provisional application No. 61/240,014, filed on Sep. 4, 2009.

(51) Int. Cl. A61K 31/23; C07C 69/30; A61P 25/24; A61P 25/22; A61P 1/00; A61P 29/00; A61P 11/00; A61P 13/10; A61P 15/00; A61P 25/00; A61P 25/30; A61P 5/00; A61P 3/00; C12N 5/071; C07C 69/33

(52) U.S. Cl. 514/552; 554/227; 435/375

(57) ABSTRACT

Compounds, pharmaceutical compositions and methods of treating a disorder or disease associated with neurokinin 2 (NK₂) receptor activity.

### CROSS-REFERENCE TO RELATED APPLICATIONS

This application claims the benefit of U.S. Provisional Application No. 61/240,014, filed Sep. 4, 2009, which is hereby incorporated by reference.

### FIELD OF THE INVENTION

The invention relates to compounds, pharmaceutical compositions and methods for treating disorders or diseases associated with neurokinin 2 (NK₂) receptor activity.

### BACKGROUND OF THE INVENTION

### Depressive Mood Disorders

Depressive mood disorders are a group of mood disorders characterized by feelings of depression. Depressive mood disorders include major depressive disorder, dysthymic disorder, depressive phase of bipolar disorder, depression due to a general medical condition such as depression associated with dementia or schizoaffective disorder, substance-induced depression, postpartum depression and seasonal affective disorder.

Major depressive disorder (also known as major depression, clinical depression, unipolar depression and unipolar disorder) is very prevalent in the general population. Recent North American data show a 14.5% lifetime risk of major depression in adults and 8.1% one year prevalence (Results from the 2004 National Survey on Drug Use and Health: National findings; Revisions as of Sep. 8, 2005; Department of Health and Human Services. Substance Abuse and Mental Health Services Administration Office of Applied Studies).

The mean duration of a depressive episode with modern treatments is about 16 weeks, although some data suggest a longer duration of about 6-8 months, far less than in the pre-antidepressant therapy era when the duration was about 18 months (Kendler, McLeod, Patten).

Antidepressants have had some impact on the treatment of major depressive disorder and on reducing the suffering of patients. Not all of the impact has been positive. Patients with major depressive disorder are often impaired in function and frequently have co-morbid disorders such as substance abuse that can be attributed to the underlying major depressive disorder. Major depressive disorder leads to increased utilization of health services and can have a devastating impact on social structure and societal economics.

The cause of major depressive disorder is not fully known. Disturbance of monoamine synthesis and activity has been a prominent etiological theory of major depressive disorder for the past few decades and support for this has been strengthened by the effectiveness of medications that enhance monoamine activity, particularly those which are serotonergic and/or noradrenergic. However, any given antidepressant is only effective in a subset of depressed patients and often only partially so. Current treatments administered in controlled trials in academic settings with selected samples show efficacy in only about 60% of patients and only about half of these have full remission of symptoms. This is important since the presence of residual symptoms is a strong predictor of relapse. There are other physiological changes associated with major depressive disorder which suggest a more complex interplay of etiological factors including the role of second messengers mediating membrane bound and intracellular processes. This has led to investigation of hormonal pathways such as the hypothalamic-pituitary-adrenal (HPA) axis (the activity of which is elevated in 20-40% of community-dwelling patients with major depressive disorder), thyroid axis (5-10% of patients evaluated with major depressive disorder have previously undetected thyroid dysfunction), growth hormone, prolactin, testosterone and the role of inflammatory processes and their markers such as interleukin-1 and -6 and tumour necrosis factor.

Most people with major depressive disorder experience some degree of symptom return, and 20-30% exhibit a chronic course (defined as a syndromal level of depressive symptom severity for two years or more (Treatment of Chronic Depression (Editorial))).

All depressed people require continuation of pharmacotherapy to permit recovery and prevent relapse. A substantial proportion of depressed patients require maintenance pharmacotherapy to prevent recurrence and further consolidate psychosocial recovery. However, while one of the major factors in effective antidepressant therapy is maintaining the patient on an adequate dose of medication for an adequate duration, this is often difficult. Many patients fear taking current antidepressants because of real or imagined physical effects. Some patients prefer to use so-called natural health promoting substances and non-pharmacological interventions. Patients who are prepared to take antidepressants often encounter a wide array of side effects, which leads them to be non-compliant or to reject therapy entirely. Selective serotonin reuptake inhibitors (SSRI) for example, commonly induce gastrointestinal upset, headaches, sleep disturbance and significant sexual impairments among many other side effects. Most antidepressants have at least some significant side effects and these limit clinicians' capacity to effectively treat many patients.

Major depressive disorder can be associated with other disorders and/or syndromes, including disorders of the brain or nervous system, anxiety disorder (which includes generalized anxiety disorder, panic disorder, phobias, obsessive-compulsive disorder, post-traumatic stress disorder, separation anxiety, social anxiety disorder, otherwise known as social phobia, bipolar disorder, and dementia); sexual dysfunction; substance abuse, eating disorders and hormone disorders, such as thyroid dysfunction, hypogonadism, menopause, etc. Treatment of the major depressive disorder often leads to improvement in these related disorders and syndromes.

In addition, some therapeutic agents used to treat depression are also effective in treating other conditions. For example, antidepressants have been demonstrated to be effective in the treatment of hot flashes associated with menopause, pain and smoking cessation.

Anxiety Disorder

Anxiety disorder is a group of disorders which affect behaviour, thoughts, emotions and physical health. Anxiety disorder is believed to be caused by a combination of biological factors and an individual's personal circumstances. People suffering from anxiety disorder are subject to intense, prolonged feelings of fright and distress for no obvious reason. The condition turns their lives into a continuous journey of unease and fear and can interfere with their relationships with family, friends and colleagues.

Anxiety disorder is among the most common of all mental health problems. It is estimated that it affects approximately 1 in 10 people. It is more prevalent among women than among men, and affects children as well as adults. It is common for people to suffer from more than one type of anxiety within the category of anxiety disorder, and for an anxiety disorder to be accompanied by depression, eating disorders and/or substance abuse.

Types of anxieties falling within the category of anxiety disorder include panic disorder (in which panic attacks occur without warning, accompanied by sudden feelings of terror and physical symptoms including chest pain, heart palpitations, shortness of breath, dizziness, abdominal discomfort, feelings of unreality and fear of dying) and social and specific phobias (the former involving a paralysing, irrational self-consciousness about social situations and the latter involving specific phobias, such as unreasonable fear of flying, blood or heights).

Another type of anxiety disorder is post-traumatic stress disorder, which may be caused by a terrifying experience in which serious physical harm occurred or was threatened. Survivors of rape, child abuse, war or a natural disaster may develop post-traumatic stress disorder. Common symptoms include flashbacks, during which the person re-lives the terrifying experience, nightmares, depression and feelings of anger or irritability.

Obsessive compulsive disorder is another type of anxiety disorder. This is a condition in which people suffer from persistent unwanted thoughts (obsessions) and/or rituals (compulsions) which they find impossible to control. Typically, obsessions concern contamination, doubting (such as worrying that a household appliance hasn't been turned off) and disturbing sexual or religious thoughts. Compulsions include washing, checking, organizing and counting.

Generalized anxiety disorder is another type of anxiety disorder, in which a person has repeated, exaggerated worry about routine life events and activities. This disorder often lasts many months, during which time the person is affected by extreme worry more days than not. The individual anticipates the worst, even if others would say he or she has no reason to expect it. Physical symptoms can include nausea, trembling, fatigue, muscle tension and/or headache.

There are two main medical approaches to treating an anxiety disorder: (1) drug therapy and (2) cognitive-behavioural therapy (CBT). Combining the two types of treatment can also be effective. Because most anxiety disorders have at least some biological component, anti-depressants and anti-anxiety drugs are generally prescribed.

Inflammatory Bowel Disease

Inflammatory bowel disease (IBD) is a group of inflammatory conditions of the colon and small intestine. The major types of IBD are Crohn's disease and ulcerative colitis. IBDs may present with any of the following symptoms: abdominal pain, vomiting, diarrhea, hematochezia (bright red blood in stools) and weight loss. Diagnosis is generally by colonoscopy with biopsy of pathological lesions.

While IBD can limit quality of life because of pain, vomiting, diarrhea, and other socially unacceptable symptoms, it is rarely fatal on its own. Fatalities due to complications such as toxic megacolon, bowel perforation and surgical complications are also rare. IBD patients do have an increased risk of colorectal cancer, although these patients are generally monitored for this on a regular basis and so colorectal cancer is usually detected much earlier than in the general population.

The treatment of IBD depends on the severity of the particular condition. IBD may require immunosuppression or a form of mesalamine. Often, steroids are used to control disease flare-ups. TNF inhibitors can also be used for both Crohn's disease patients and patients with ulcerative colitis. Severe cases may require surgery, such as bowel resection, strictureplasty or a temporary or permanent colostomy or ileostomy.

The goal of treatment is to achieve remission, after which the patient is usually switched to a less potent drug with fewer potential side effects. Occasionally, an acute resurgence of the original symptoms may appear. Depending on the circumstances, it may go away on its own or require medication. The time between such resurgences may be anywhere from weeks to years, and varies widely between patients.

Irritable Bowel Syndrome

Irritable bowel syndrome (IBS) is a disorder characterized most commonly by cramping, abdominal pain, bloating, constipation and/or diarrhea. IBS causes a great deal of discomfort and distress, but it does not permanently harm the intestines and does not lead to a serious disease, such as cancer. Many people can control their symptoms with diet, stress management, and prescribed medications. For some people, however, IBS can be disabling. They may be unable to work, attend social events, or even travel short distances.

As many as 20 percent of the adult population have symptoms of IBS, making it one of the most common disorders diagnosed by doctors. It occurs more often in women than in men, and it begins before the age of 35 in about 50 percent of those affected. Sometimes people find that their symptoms subside for a few months and then return, while others report a constant worsening of symptoms over time.

There is no specific diagnostic test for IBS, although diagnostic tests may be performed to rule out other problems. These tests may include stool sample testing, blood tests, and x rays. Typically, a doctor will perform a sigmoidoscopy or a colonoscopy. The doctor may diagnose IBS based on the patient's symptoms, including frequency of abdominal pain or discomfort during the past year, when the pain starts and stops in relation to bowel function, and how bowel frequency and stool consistency have changed.

Unfortunately, many people suffer from IBS for a long time before seeking medical treatment. Up to 70 percent of people suffering from IBS are not receiving medical care for their symptoms. Medications are an important part of relieving the symptoms of IBS. Such medications include fiber supplements or laxatives for constipation, medicines to decrease diarrhea and antispasmodics to control colon muscle spasms and reduce abdominal pain. In addition, antidepressants may relieve some symptoms of IBS.

Inflammatory Airway Disease

Inflammatory airway disease includes asthma and chronic obstructive pulmonary disease (COPD). Asthma is a chronic inflammation of the lungs in which the airways (bronchi) are reversibly narrowed. Asthma affects 7% of the population and 300 million people worldwide. During an asthma attack, the smooth muscle cells in the bronchi constrict, and the airways become inflamed and swollen. This results in difficulties in breathing.

Asthma causes approximately 4,000 deaths a year in the U.S. Attacks can be prevented by avoiding triggering factors and by drug treatment. Drugs such as inhaled β2 agonists are often used for acute attacks. In more serious cases, drugs are used for long-term prevention, starting with inhaled corticosteroids, and then long-acting β2-agonists if necessary. Leukotriene antagonists can also be used in place of corticosteroids. Monoclonal antibodies such as mepolizumab and omalizumab are sometimes effective.

COPD includes a few lung diseases such as chronic bronchitis and emphysema. Many people with COPD have both of these diseases. Symptoms of COPD include shortness of breath, increased mucus in the lungs and coughing. The main treatments for COPD are: quitting smoking, medications, such a bronchodilators and corticosteroids and pulmonary rehabilitation.

Urinary Incontinence

Urinary incontinence is the inability to control the release of urine from the bladder. Some people experience occasional, minor leaks of urine, while others wet their clothes frequently. Types of urinary incontinence include stress incontinence, urge incontinence and overflow incontinence. Treatment for urinary incontinence depends on the type of incontinence, the severity of the problem and the underlying cause. Treatment may include, for example, behavioural techniques, physical therapy and/or medications such as anticholinergics, topical estrogens and imipramine.

The limited efficacy, often unacceptable side effects and physiological factors that may induce or otherwise affect the course of the disorders and diseases discussed above make it necessary to continue to search for new compounds with novel pharmacological actions to address these disorders and diseases.

### SUMMARY OF THE INVENTION

The present invention features a compound having the following the structure:

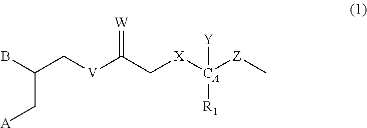

wherein:
(i) A and B are independently —OH or —SH,
(ii) V and W are independently oxygen or sulfur and at least one of V and W is oxygen,
(iii) R₁ is —(CH₂)ₚCH₃ or is —H, and
(iv) p is an integer from 0 to 3, and:
(A) X is —(CH₂)ₘ—,
(B) Y is —H,
(C) Z is —(CH₂)ₙ—,
(D) m and n are integers,
(E) m=1 to 5,
(F) n=4 to 14,
(G) 6≦m+n≦14 for all m and n, and
(H) wherein, optionally, there are up to two carbon-carbon double bonds, each double bond formed between adjacent methylene groups of formula (1) wherein, if there are two said double bonds each carbon thereof is bonded to at least one hydrogen;
or (I) X is

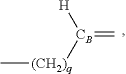

(J) Y is absent, and C_{A} and C_{B} together form a double bond,
(K) Z is —(CH₂)ᵣ—,
(L) q and r are integers,
(M) q=0 to 4,
(N) r=1 to 13,
(O) 5≦q+r≦13 for all q and 4, and
(P) wherein, optionally, there is a second double bond formed between adjacent methylene groups of formula (1) wherein each carbon thereof is bonded to at least one hydrogen;
or (O) X is —(CH₂)ₜ—,
(R) Z is

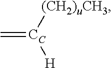

(S) Y is absent, and C_{A} and C_{C} together form a double bond,
(T) R₁ is —(CH₂)ᵥCH₃ or is —H,
(U) t and u are integers,
(V) t=1 to 5,
(W) u=0 to 12,
(X) 5≦t+u≦13 for all t and u, and
(Y) wherein, optionally, there is a second double bond formed between adjacent methylene groups of formula (1) wherein each carbon thereof is bonded to at least one hydrogen,
   including a pharmaceutically acceptable salt of the compound.

In one aspect of the invention, A and B are both —OH.

V and W can both be oxygen.

Preferably, R₁ is —(CH₂)ₚCH₃. The value of p can be from 0 to 2, more preferably p is 0 or 1, and most preferably p is 0.

The value of n can be from 2 to 12 while 7≦m+n≦13, or n can be 3 to 11 and 8≦m+n≦12, or n can be from 4 to 10 and 9≦m+n≦11, more preferably, n is 5 to 9 and m+n=10. The value of m can be from 2 to 4, but is preferably 3.

The value of r can be from 2 to 12 while 6≦q+r≦12, more preferably r is from 3 to 11 while 7≦q+r≦11, more preferably r is from 4 to 10 while 8≦q+r≦10, and most preferably, r is from 5 to 9 and q+r is 9.

The value of q can be from 1 to 3 and preferably q is 2.

The value of u can range from 1 to 11 while 6≦t+u≦12, more preferably u is from 2 to 10 and 7≦t+u≦11, more preferably u is from 3 to 9 while 8≦t+u≦10 and most preferably, u is 4 to 8 and t+u is 9.

The value oft can be from 2 to 4 and is preferably 3.

If one or both of two carbon-carbon double bonds of foregoing paragraph (H) are present in the compound, then each of those bonds can be formed between methylene groups of Z. A methylene group is —(CH₂)—. Preferably, if such a bond is present, there is only one of them.

If the second double bond of paragraph (P) is present, then the bond is preferably formed between methylene groups of Z.

Most preferably, none of the double bonds of paragraphs (H) and (P) are present.

A preferred compound has Formula (I), this compound being a mixture of all four stereoisomers. When stereoisomers are referred to herein, we are speaking of those that result from the presence of two chiral centers such as those found in the compound of Formula (I) and discussed further below.

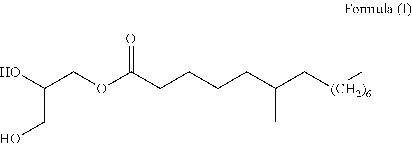

A preferred compound is a substantially stereochemically pure compound of Formula (I) of the structure:

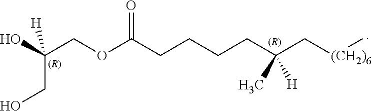

Another preferred compound is a substantially stereochemically pure compound of Formula (I) of the structure:

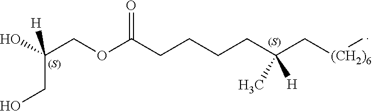

Another preferred compound is a substantially stereochemically pure compound of Formula (I) of the structure:

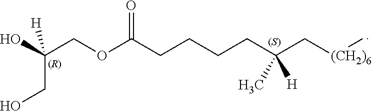

Another preferred compound is a substantially stereochemically pure compound of Formula (I) of the structure:

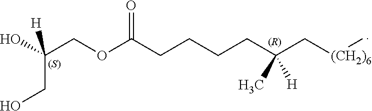

Throughout this specification Formula (I) is often referred to as 6-methyl myristic acid monoglyceride, more often 6-MMAM. When a compound of the invention is referred to as having Formula (I) with no other descriptors, this means that the compound is a mixture of the four stereoisomers described above.

The invention includes a compound of Formula (I) in which the chiral carbon of the glycerol moiety is a mixture of R and S stereochemical configurations while the C-6 carbon of the myristic acid moiety is R. Also included is a compound of Formula (I) in which the chiral carbon of the glycerol moiety is a mixture of both R and S stereochemical configurations while the C-6 carbon of the myristic acid moiety has the S configuration. Further, the invention includes a compound of Formula (I) in which the chiral carbon of the glycerol moiety has the S configuration while the C-6 carbon of the myristic acid moiety is a mixture of R and S. Also included is a compound of Formula (I) in which the chiral carbon of the glycerol moiety has the R configuration while the C-6 carbon of the myristic acid moiety is a mixture of R and S.

The invention includes a pharmaceutical composition containing any of the compounds described above.

The pharmaceutical composition can be adapted for oral delivery, parenteral delivery, topical delivery, rectal delivery, vaginal delivery, administration by oral inhalation or nasal delivery.

The invention includes any of the foregoing compounds in various forms. Particular dosage forms include a solution, a suspension, a syrup, a tablet, a capsule, microparticles, an ointment, a cream or a lozenge, or a capsule, with a preferred form being a tablet.

The invention includes a method for treating a disorder or disease associated with neurokinin 2 (NK₂) receptor activity. The method includes the step of administering a therapeutically effective amount of any of the foregoing compounds. When compounds of the invention are referred to herein, it is to be understood that this includes pharmaceutically acceptable salts, whether explicitly stated or not.

Disorders or diseases associated with said NK₂ receptor activity treated according to methods of the invention can be a depressive mood disorder, anxiety disorder, irritable bowel syndrome, inflammatory bowel disease, inflammatory airway disease or urinary incontinence. The disorder or disease associated with said NK₂ receptor activity can specifically be depressive mood disorder, or it can be major depressive disorder.

The subject or patient may or may not also be treated by psychotherapy concurrently with a method of the invention.

Of course a compound of the invention can be contained in a pharmaceutical formulation that also includes a pharmaceutically acceptable carrier.

Administration of a compound described herein can be accompanied by a therapeutically effective amount of another therapeutic agent.

Typically, subjects treated using the invention are human patients.

Another method of the invention is for treating a disorder or syndrome associated with a depressive mood disorder. The method includes the step of administering a therapeutically effective amount of a compound of the invention to a subject in need thereof. The disorder or syndrome can be a disorder of the brain or nervous system, anxiety disorder, sexual dysfunction, substance abuse, eating disorder or hormone disorder.

In another aspect, the invention is a method of treating a disorder or condition treatable by an antidepressant. The method includes administering a therapeutically effective amount of a compound of the invention a subject in need thereof. The disorder or condition treatable by an antidepressant can be hot flashes associated with menopause, pain or smoking cessation.

Another method of the invention is for modulating an activity of an NK₂ receptor comprising contacting the NK₂ receptor with an effective amount of a compound of the invention. The method can be an in vivo or an in vitro method.

The invention includes use of a compound, or a pharmaceutically acceptable salt thereof, described above for treatment of a disorder or disease, etc. as described above in connection with various methods of the invention.

An inventive use of a compound of the invention is thus also in the manufacture of a medicament for treatment of such a disorder or disease, etc.

### BRIEF DESCRIPTION OF THE FIGURES

The person skilled in the relevant art(s) will understand that the figures, described below, are for illustration purposes only. The figures are not intended to limit the scope of the invention in any way.

FIG. 1 shows an HPLC chromatogram of a fertilized egg isolate according to embodiments of the present invention.

FIG. 2 shows the results of analyses of a fertilized egg isolate according to embodiments of the present invention.

FIG. 3 shows a graph of the effect of various concentrations of a fertilized egg isolate Sample #20 Top Isolate (μg/mL) on binding of neurokinin A (NKA) to the human NK₂ receptor (measured as percent of specific binding) as well as the IC₅₀ and Kᵢ for NKA and Sample #20 Top Isolate.

FIG. 4 shows a bar graph of the binding activity of various fractions and a control sample of Formulation A (eluted from an HPLC) to the human NK₂ receptor. Binding activity was measured as the percent inhibition of binding by the ligand NKA.

FIG. 5 shows a chromatogram from an HPLC-UV of Fraction 171 of Formulation A. The UV detector was set at 210 nm. The units along the x-axis are time (minutes) and the units along the y-axis are absorbance units (AU).

FIG. 6 shows a chromatogram from an HPLC-UV of Fraction 185 of Formulation A. The UV detector was set at 210 nm. The units along the x-axis are time (minutes) and the units along the y-axis are absorbance units (AU).

FIG. 7 shows a chromatogram from an HPLC-UV of Fraction 171 of Formulation A. The UV detector was set at 190 nm. The units along the x-axis are time (minutes) and the units along the y-axis are absorbance units (AU).

FIG. 8 shows a chromatogram from an HPLC-UV of Fraction 185 of Formulation A. The UV detector was set at 190 nm. The units along the x-axis are time (minutes) and the units along the y-axis are absorbance units (AU).

FIG. 9 shows a graph of the effect of various concentrations of 6-methyl-myristic acid 2,3-dihydroxypropyl ester on binding of neurokinin A (NKA) to the human NK₂ receptor (measured as percent of specific binding) as well as the IC₅₀ and Kᵢ for NKA and 6-methyl-myristic acid 2,3-dihydroxypropyl ester.

FIG. 10 shows a graph of the effect of various concentrations of myristic acid 2,3-dihydroxypropyl ester on binding of neurokinin A (NKA) to the human NK₂ receptor (measured as percent of specific binding) as well as the IC₅₀ and Kᵢ for NKA and myristic acid 2,3-dihydroxypropyl ester.

FIG. 11 shows a graph of the effects of bAla⁸-NKA(4-10) (control), compound #2 (myristic acid 2,3-dihydroxypropyl ester) and compound #3 (6-methyl-myristic acid 2,3-dihydroxypropyl ester) in a cellular/functional Ca²⁺ agonist assay for the human NK₂ receptor. The x-axis indicates the log of the compound (M) and the y-axis indicates the % maximal response (RFU). Error bars are ranges for duplicate data.

FIG. 12 shows a graph of the effects of bAla⁸-NKA(4-10) (control), GR159897 (control), compound #2 (myristic acid 2,3-dihydroxypropyl ester) and compound #3 (6-methyl-myristic acid 2,3-dihydroxypropyl ester) in a cellular/functional Ca²⁺ antagonist assay for the human NK₂ receptor. The x-axis indicates the log of the compound (M) and the y-axis indicates the % maximal response (RFU). Error bars are ranges for duplicate data.

### DETAILED DESCRIPTION OF THE INVENTION

In accordance with the present invention, the compounds of the invention and uses to treat disorders or diseases associated with neurokinin (NK₂) receptor activity are described.

As used herein, the term “pharmaceutically acceptable salt” is a salt formed from an acid and a basic group of a compound having a structural formula of the invention. Illustrative salts are known to one skilled in the art and include, but are not limited, to hydrochloride, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

According to this invention, the chemical structures depicted herein, including the compounds of this invention, encompass all of the corresponding compounds' enantiomers and stereoisomers, that is, both the stereomerically pure form (e.g., geometrically pure, enantiomerically pure, or diastereomerically pure) and enantiomeric, diastereomeric, and geometric isomeric mixtures and as such, are also compounds of the invention. Methods for separating one enantiomer from another are known to those skilled in the art. In some cases, one enantiomer, diastereomer, or geometric isomer will possess superior activity or an improved toxicity or kinetic profile compared to others. In those cases, such enantiomers, diastereomers, and geometric isomers of a compound of this invention are preferred.

The compounds of the invention, including any enantiomers of such compounds, may be substantially pure. A compound is “substantially pure” when it is separated from the components that naturally accompany it. Thus, for example, a compound of Formula (I) that is isolated from fertilized egg isolate will generally be substantially pure when it is separated from other components of the fertilized egg isolate. Typically, a compound is substantially pure when it is present in at least 60%, 70%, 75%, 80%, 85%, 90%, 95% or 99%, by weight, of the total material in a sample. A substantially pure compound can be obtained, for example, by extraction from a natural source, such as a fertilized egg isolate or by chemical synthesis. Purity can be measured using any appropriate method such as column chromatography, gel electrophoresis, high pressure liquid chromatography (HPLC), etc.

The compounds of the invention can be used to treat a disorder or disease associated with NK₂ receptor activity. A compound of the invention is administered in a therapeutically effective amount to a subject in need thereof.

The term “treat” means improving the disorder or disease of a patient to whom a compound of the present invention is being administered. The term “treatable” means capable of improving the disorder, disease or condition of a patient to whom a compound of the present invention is being administered. These term include ameliorating the disorder, disease or condition, for example, by obtaining a beneficial outcome, and such amelioration can be determined using standard tests known in the art. The terms also include preventing the disorder or disease from occurring or re-occurring, such as in prophylactic or maintenance therapy.

As used herein, the term “NK₂ receptor-associated disorder or disease” refers to a disorder or disease associated with inappropriate, e.g., greater or less than normal, NK₂ receptor activity. The greater than normal NK₂ receptor activity may result from increased activity of a normal number of NK₂ receptors in the subject, or could result from a greater than normal number of NK₂ receptors in the subject with the NK₂ receptor-associated disorder or disease. The less than normal NK₂ receptor activity may result from decreased activity of a normal number of NK₂ receptors in the subject, or could result from a less than normal number of NK₂ receptors in the subject with the NK₂ receptor-associated disorder or disease. NK₂ receptor-associated disorders or diseases include, for example, major depressive disorder, anxiety disorder, irritable bowel syndrome, inflammatory bowel disease, inflammatory airway disease and urinary incontinence. An NK₂ receptor-associated disorder or disease may include a disorder or disease that is mediated, at least in part, by an NK₂ receptor.

An “effective amount” is the quantity of compound in which a beneficial outcome is achieved when the compound is administered to a subject with a disorder or disease associated with NK₂ receptor activity or alternatively, the quantity of compound that possesses a desired activity in vivo or in vitro. In the case of a disorder or disease associated with NK₂ receptor activity, a beneficial outcome includes reduction in the extent or severity of the symptoms associated with the disease or disorder and/or an increase in the quality of life of the subject compared with the absence of the treatment. For example, for a subject with major depressive disorder, a “beneficial outcome” includes a decrease in the rating of a subject on the Hamilton Depression Rating Scale, the Hamilton Anxiety Rating Scale, the Montgomery-Åsberg Depression Rating Scale, the Beck Depression Inventory, the Arizona Sexual Experience Scale, or the General Health Questionnaire Scoring (Short-Form 36), each of which is known to one skilled in the art and is described in further detail herein as compared to the rating in the subject who has not been treated with a compound of the invention.

For a subject with anxiety disorder, a “beneficial outcome” includes a decrease in the rating of a subject on the Hamilton Anxiety Rating Scale, decreased feelings or decreased frequency of feelings of distress and fright, decreased number and/or duration of panic attacks, decreased avoidance of social situations, decreased fears associated with specific phobias, decreased occurrence and duration of flashbacks, nightmares, depression and feelings of anger or irritability associated with post-traumatic stress disorder and decreased occurrence of obsessions and/or compulsions as compared to the subject who has not been treated with a compound of the invention.

For a subject with inflammatory bowel disease, a “beneficial outcome” includes decreased abdominal pain, vomiting, diarrhea, hematochezia and/or weight loss as compared to a subject who has not been treated with a compound of the invention.

For a subject with irritable bowel syndrome, a “beneficial outcome” includes a decrease in cramping, abdominal pain, bloating, constipation, and/or diarrhea as compared the subject who has not been treated with a compound of the invention.

For a subject with inflammatory airway disease, a “beneficial outcome” includes decreased shortness of breath, decreased mucus in the lungs and/or decreased frequency and/or duration of coughing spells as compared to the subject who has not been treated with a compound of the invention.

For a subject with urinary incontinence, a “beneficial outcome” includes decreased leaking or urine and/or wetting of clothes as compared to the subject who has not been treated with a compound of the invention.

The precise amount of compound administered to a subject will depend on the type and severity of the disorder or disease and on the characteristics of the subject, such as general health, age, sex, body weight and tolerance to drugs. The skilled artisan will be able to determine appropriate dosages depending on these and other factors.

As used herein, the terms “subject”, “patient” and “animal” are used interchangeably and include, but are not limited to, a cow, monkey, horse, sheep, pig, chicken, turkey, quail, cat, dog, mouse, rat, rabbit, guinea pig and human. In one embodiment, the subject, patient or animal is a mammal. In another embodiment, the preferred subject, patient or animal is a human.

### Methods for Isolating a Compound of the Invention:

A compound having the structure depicted by Formula (I) can be isolated from fertilized egg isolates as described below.

Fertilized Egg Isolates—Preparation

In the preparation of a fertilized egg isolate from which the compound of Formula (I) can be isolated, at least one fertilized egg is incubated for anywhere from about 3 to about 15 days, more preferably about 3 to about 5 days, or more preferably about 6 to about 12 days, and even more preferably about 7 to about 9 days, from the day the egg is fertilized. Generally speaking, the fertilized egg is incubated for a period of time that allows angiogenesis to initiate and/or the embryo to mature to the point that embryos are visible to the naked eye. The eggs can be from a variety of origins, for example, avian, reptilian, or from egg-laying mammals. Generally speaking, any egg from which an embryo or blood vessels associated with an embryo can be removed can be suitable. The eggs are preferably avian eggs, and can be obtained from any bird that has been bred for egg production, such as chicken, geese, ducks, and the like. Chicken eggs are preferred for reasons including their availability and ability to be mass produced. Incubation can occur in any environment, so long as the eggs are kept at a temperature for extended periods of time that allows maturation of the embryo. Suitable temperatures for incubation are in the range of about 20° C. to about 60° C., more preferably in the range of about 25° C. to about 55° C., and more preferably in the range of about 35° C. to about 45° C. Once the eggs are incubated for a period of time, they are optionally treated to reduce external microflora or otherwise sterilized by any suitable means, such as washing the egg shells with a solvent such as ethanol, for example, an about 50% to about 95% solution of ethanol, with subsequent time allowed to permit evaporation or drying of the solvent, or by rotating the eggs under an ultraviolet (UV) light source for a suitable period of time. Any solvent is preferably evaporated before further manipulation of the egg. The eggs are then cracked to access the inner contents. The eggs can be cracked under aseptic conditions either manually or using a suitable mechanical device. This procedure and/or all or most of the procedures described above and below can be conducted in a cooled atmosphere, such as an atmosphere of about 5° C.

The contents of the egg are collected in a container, such as a stainless steel container, which is preferably sterilized and/or chilled. The contents from the container or from the egg can optionally be subjected to a filtration process, for example, by being placed on a mesh. The mesh openings can be about 0.5 to about 4 millimeters, more preferably about 1 millimeter. The mesh is preferably sterile.

Optionally, the contents of the egg and/or some or all of the broken shell can be placed directly on the mesh. The contents of the egg and/or some or all of the broken shell are allowed to filter on the mesh for a period of time such that there is substantially no further dripping of fluid through the mesh. The broken shell can be removed from the contents of the egg before, during, or after the filtration process. After the filtration, the solid or solid and semi-solid retentate can comprise the embryo, vascular connective tissue, a substantial portion or all of the albumen, a substantial portion or all of the chalaza, and the clear sac. Semi-solid retentate can comprise solid material as well as a viscous material, such as a gelatinous material, for example, albumen. The retentate or semi-solid retentate can be optionally washed at least once with a suitable solvent, such as a buffer solution, sterile deionized water, or any suitable saline solution. For example, sterile phosphate buffer saline (PBS) can be used.

The retentate can be collected from one egg and then freeze-dried according to the processes described herein, or the retentate can be collected from one or more eggs together, and then freeze-dried according to the processes described herein.

The white albumen portion and/or embryo can be substantially separated from the rest of the contents of the egg. The white albumen portion may be substantially separated from the rest of the contents by any suitable means, such as decantation of the white albumen portion, or by suction. The embryo can be substantially separated from the white albumen portion manually or other suitable means as determined by the skilled person. It will be recognized by those skilled in the art that the embryo can be substantially separated from the white albumen portion and the rest of the inner contents at the same time. For example, the embryo can be manually removed from the white albumen portion and rest of the inner contents using tweezers or other suitable instrument. In some cases, the embryo can be manually peeled off the yolk sac, which forms part of the rest of the inner contents.

Once the embryo is substantially separated from the white albumen portion and the rest of the inner contents of the egg, the embryo is optionally washed at least once with a suitable solvent, such as a buffer solution, sterile deionized water, or any suitable saline solution. For example, sterile phosphate buffer saline (PBS) can be used.

It will be understood for the following methods that reference to contents of the egg may actually be a reference to the retentate if the contents have been subjected to a filtration process. It will also be understood that a whole fertilized egg can be cracked, the shell removed, and the whole of the shelled egg frozen and freeze-dried according to any of the procedures described above and below, to produce a fertilized egg isolate. Also, more than one whole fertilized egg can be cracked, shells removed, the whole of the shelled fertilized eggs combined and blended into a slurry, and frozen and freeze-dried according to any of the procedures described above and below.

The contents of the eggs or the embryos are placed in at least one freezable container. The container can be, for example, a test tube, Petri dish, beaker, stainless steel tray, or plastic container. It is preferred that the contents or embryos are frozen very soon after being removed from the shell, such as within about 2 hours, more preferably within about 1 hour, and even more preferably within about 0.5 hours, or as soon as possible. Depending on how long the contents or embryos are to be frozen, the freezing temperature should be in the range of about −50° C. to about 10° C., more preferably in the range of about −40° C. to about 5° C., and even more preferably in the range of about −35° C. to about −25° C. It is preferred that the contents or embryos are frozen for at least about 6 hours, more preferably at least about 12 hours, even more preferably at least about 24 hours. The frozen contents or embryos may be freeze-dried or lyophilized after a period of time. The contents or embryos can be completely frozen before the freeze-drying/lyophilizing step.

Optionally, frozen or unfrozen contents or embryos can be pooled in a suitable container, such as a beaker, or a plastic container, and mixed or blended with a suitable solvent, if necessary, to form a slurry. The solvent can be suitably aqueous to wet the mixed contents or embryos and be able to be frozen in a standard laboratory freezer. Suitable solvents include water, aqueous buffer, and the like. To form the slurry, it is preferred that the contents and/or embryos are blended. The contents or embryos can be blended or homogenized with, for example, a hand-held blender or other suitable means. The slurry can then be frozen as described above and freeze-dried. Freeze-drying is preferably performed at an ultimate temperature in the range of about −80° C. to about −10° C., more preferably in the range of about −65° C. to about −15° C., and even more preferably in the range of about −40° C. to about −20° C. and a pressure of about 500 millitorr, or other suitable pressure as can be determined by the skilled person. The freeze-drying process is preferably maintained at the ultimate temperature for a period of in the range of about 1 to about 6 hours, more preferably in the range of about 2 to about 5 hours, and even more preferably in the range of about 3 to about 4 hours. The whole freeze-drying process is typically conducted for a period in the range of about 15 to about 45 hours, more typically in the range of about 25 to about 35 hours, and even more typically in the range of about 28 to about 32 hours.

The freeze-dried contents, freeze-dried embryo, or freeze-dried slurry is then dispersed and/or pulverized if necessary to form a substantially homogeneous powder. The contents that were freeze-dried individually or in smaller groups can be combined together before or after the pulverization step to form a substantially homogeneous powder. The pulverization can be done, for example, mechanically using a suitable machine, such as a coffee bean grinder or a hammer mill, or manually using a suitable tool, such as a glass rod. A suitable sterilization should be one that does not adversely affect certain freeze-dried components.

In association with any process described herein, preservatives to control microbial growth can be blended into the powder or concentrate before it is stored. Preservatives can also be added at another stage of the manufacture, including before the freeze-drying or concentration step instead of, or in addition to, being added to the powder or concentrate. Suitable preservatives include common food preservatives such as 0.5% w/w sodium benzoate and 0.2% w/w potassium sorbate. Other suitable preservatives could be selected by the skilled person.

The powders produced by the processes disclosed herein can be stored in suitable, substantially air-tight containers. Suitable containers include plastic bags, barrels, plastic containers, bottles, combinations thereof, and the like. For example, the powder can be packaged under controlled, aseptic conditions into sterile polyethylene/polypropylene bottles with tamper-proof security seals. The powder can be stored under a substantially dry, inert gas, such as nitrogen. It is preferred that the powder be stored at room temperature or cooler, for example, at a temperature in the range of about 10° C. to about 25° C., more preferably in the range of about 15° C. to about −20° C. For long term storage, it is preferred that the powder is stored at a temperature of about −10° C. or below, or, more preferably, −20° C. or below. The powder can be stored for a period of time in a substantially desiccated atmosphere. The powder can also be vacuum-packed.

A slurry can also be prepared by separating the contents or embryos of at least one fertilized egg from the egg shell, and pooling the separated contents or embryos in a suitable container. The separated contents or embryos can be cooled during this step. For example, the container can be placed on ice to facilitate cooling. The contents or embryos can be blended by methods described above to produce a slurry. The slurry can be freeze-dried as described above, or partially or wholly used for extraction procedures as follows.

The slurry may also be mixed with an aqueous solution for a period of time. The aqueous solution may comprise water, an aqueous buffer, or any other aqueous solvent. If the aqueous solution comprises water, it is preferred that the water is distilled and, more preferably, also deionized before use. For example, the water can be treated using reverse osmosis (R.O.). The slurry and the aqueous solution can be mixed, for example, by stirring for a period of time, the period of time being in the range of about 5 to about 60 minutes, more preferably in the range of about 10 to about 45 minutes, and even more preferably in the range of about 15 to about 40 minutes. It is desired that the aqueous solution has sufficient exposure to the contents of the slurry so that any substantially hydrophilic molecules in the solution are dissolved in the aqueous solution. The aqueous solution can be of a substantially equal volume to the slurry, but volumes of 1.5 times, 2 times, or even 3 times the volume of the slurry can be used. Optionally, the mixture can be warmed slightly during the mixing step. After the mixing, the aqueous solution can be substantially clarified by substantially removing any solid portions in the mixture by suitable means such as centrifugation or filtration. The clarified aqueous portion can then be frozen and freeze dried to produce a powder that is optionally sterilized according to methods described herein.

The slurry produced by any of the methods described above can be mixed with a substantially hydrophobic solvent. The substantially hydrophobic solvent is preferably chilled. Suitable hydrophobic solvents include, for example, ether, chloroform, hexane, petroleum ether or acetonitrile. For example, ether, especially diethyl ether, can be used. The slurry is mixed with the hydrophobic solvent for a period of time as described above. As will be recognized by a person skilled in the relevant arts, any steps of a process using a substantially hydrophobic solvent should be conducted in a fume hood or similar device, and the solvents should be kept away from open flames or heat sources. After the mixing period, the solid portions of the mixture can be substantially removed from the solvent portion by suitable means such as centrifugation or filtration. The solvent portion will comprise substantially a hydrophobic solvent portion and may also comprise an aqueous portion. The solvent portion can be transferred to a separating funnel or essentially equivalent device to separate the aqueous portion from the hydrophobic solvent portion. If the top layer is the hydrophobic solvent portion, it can be siphoned off the top or removed from the separating funnel after the bottom aqueous layer is removed. Alternatively, the bottom aqueous portion can be frozen, thereby allowing the top ether-based layer to be decanted. The aqueous portion can be extracted a number of times, for example, about 3 times, with the hydrophobic solvent. The hydrophobic solvent can be of substantially equal volume to the aqueous portion, or can be 1.5 times, 2 times or even 3 times the volume of the aqueous solvent. Other ratios may also be suitable.

After the extraction process, all of the hydrophobic isolates can be pooled and concentrated by a suitable method. The concentrated isolates can be stored at a temperature below room temperature, such as about 5° C. in a suitable container that is substantially sealed from the atmosphere, such as a sealed vial.

The slurry produced by any of the methods described above can be clarified before an extraction procedure. Preferred clarification steps include methods of filtration, using such filters as sieves or filter papers or pads. Other clarification steps can include methods of centrifugation. A filter aid, such as Superflow DE™ can be added to the filtrate produced by the filtering step before further clarification. Some of the resultant filtrate can be frozen in suitable containers for freeze-drying. Also, some of the resultant filtrate can be mixed with a hydrophobic solvent as described above so that an aqueous layer and a hydrophobic layer are formed. The layers can be separated, concentrated, and stored as described herein.

The fertilized egg isolate prepared by various processes described herein can be concentrated by repeated aqueous and/or hydrophobic solvent extraction.

The compound having the structure depicted by Formula (I) can be isolated from the fertilized egg isolate using, for example, standard column chromatography techniques. For example, a slurry of the fertilized egg isolate can be prepared as described above and freeze-dried. The freeze-dried product can then be pulverized in a grinder and, if desired, mixed with one or more preservatives such as sodium benzoate (e.g., 0.5% w/w) and/or potassium sorbate (e.g., 0.2% w/w). The finished powder can then be loaded onto a high pressure liquid chromatograph (HPLC) column and eluted with a suitable solvent, for example, various concentrations of methanol or acetonitrile or a mixture of solvents. The compound fraction of the eluate is collected and dehydrated, if desired, or subjected to additional rounds of column chromatography, using, for example, a different column, a different solvent or a different concentration of solvent.

The purity of the desired fractions can be monitored using, for example, HPLC, or other methods known to those skilled in the art and the fraction can be further purified, if desired, using techniques known to those skilled in the art.

Once a fraction or a combination of fractions is sufficiently pure, the structure of the active compound and its biological activity can be confirmed using methods known to those skilled in the art. For example, the biological activity of the active compound can be evaluated using NK₂ receptor binding assays and/or receptor activity assays known to those skilled in the art.

### Synthesis of Compounds of the Invention

A stereoisomeric mixture of the preferred compound of the invention, 6-methyl myristic acid monoglyceride, was synthesized according to Scheme A.

Racemic 2-methyldecanal [19009-56-4] was thus allowed to react with triphenylposphonium butanoic acid bromide [17857-14-6] to afford after purification 6-methyl-4-ene-tetradecanoic acid. The analogous acid chloride was prepared with thionyl chloride and treated directly with racemic isopropylidene glycerol then subjected to hydrogenation. The isopropylidene was removed with HCl to obtain 6-methyl myristate 1-glyceride as a mixture of stereoisomers.

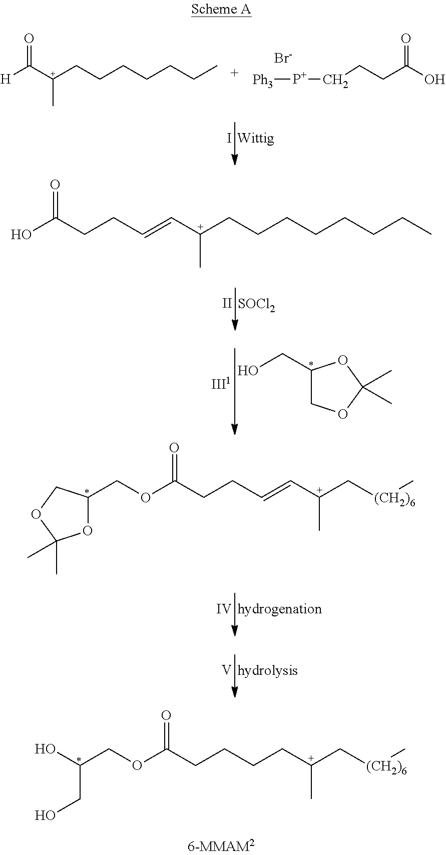

1. The dioxolane of Step III is available from Sigma-Aldrich, St. Louis, Mo., U.S.A., or can be prepared according to the route shown in Scheme B.
2. For brevity, 6-methyl myristic acid monoglyceride is referred to herein as 6-MMAM. 6-MMAM is also known as 6-methyl myristate 1-glyceride and 6-methyl glycidyl myristate.

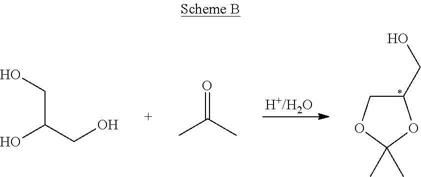

6-MMAM has two chiral centers and therefore exists as four stereoisomers:

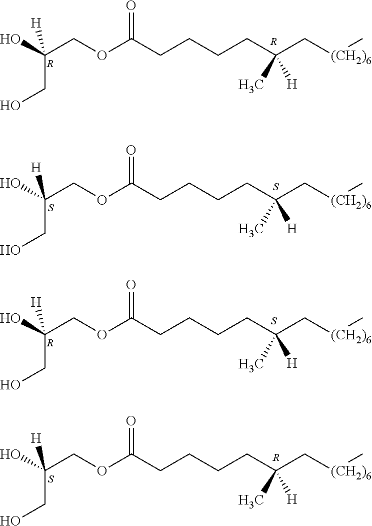

The synthetic route shown in Scheme A produces a mixture of all four stereoisomers, but can be modified to obtain stereoisomers of 6-MMAM.

Step III of Scheme A can thus be modified by use of either of the stereoisomers (R- or S-isomer at C*) of the 2,2-dimethyl-1,3-dioxolane shown, or other suitable 1,3-dioxolane. U.S. Pat. No. 6,143,908, Hinoue et al., describes a process for preparation of 1,3-dioxolane-4-methanol compounds according to Scheme C.

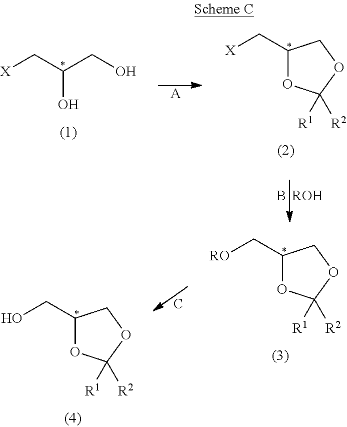

Hinoue et al. state that preferred examples of compound (I) of Scheme C are 3-chloro-1,2-propanediol and 3-bromo-1,2-propanediol, and that R¹ and R² of the compound can be the same or different and be hydrogen, C₁-C₄ alkyl, or phenyl. The dioxolane introduced in Step III of Scheme A corresponds to that in which R¹ and R² are both methyl groups in Scheme C. In other words, the use of acetone in Step A of Scheme C will result in the formation of the dioxolane shown in Scheme A. Hinoue et al. demonstrate the preparation of (S)-2,2-dimethyl-1,3-dioxolane-4-methanol using (R)-3-chloro-1,2-propanediol as the starting compound in Scheme C.

(R)-3-chloro-1,2-propanediol (CAS No. 57090-45-6) and (S)-3-chloro-1,2-propanediol (CAS No. 60827-45-4) are available from TCI America, 9211 N. Harborgate Street, Portland, Oreg. 97203, U.S.A, so these could be used to produce (S)-2,2-dimethyl-1,3-dioxolane-4-methanol and (R)-2,2-dimethyl-1,3-dioxolane-4-methanol.

(R)-4-chloromethyl-2,2-dimethyl-1,3-dioxolane (CAS No. 57044-27-3) and (S)-4-chloromethyl-2,2-dimethyl-1,3-dioxolane (CAS No. 60456-22-6) are available from Ivy Fine Chemicals Corporation of 1879 Old Cuthbert Road, Suite 23, Cherry Hill, N.J. 08034, USA. Either of these could be introduced in Step B of Scheme C, the Hinoue et al. process, for R¹=R²=methyl rendering unnecessary Step A of Scheme C.

Examples of hydrolysis of 1,3-dioxolanes under mild conditions, Step III of Scheme A, are described, for example, by J. Sun, Y. Dong, L. Cao, X. Wang, S. Wang, Y. Hu, J. Org. Chem., 2004, 69:8932-8934 and R. Dalpozzo, A. De Nino, L. Maiuolo, A. Procopio, A. Tagarelli, G. Sindona, G. Baroli, J. Org. Chem., 2002, 67:9093-9095.

The second chiral center (C⁺) in 6-MMAM is introduced in the first step of Scheme A, 2-methyldecanal.

The synthesis of Scheme A can be varied to obtain other compounds of Formula (1), as shown in Scheme D.

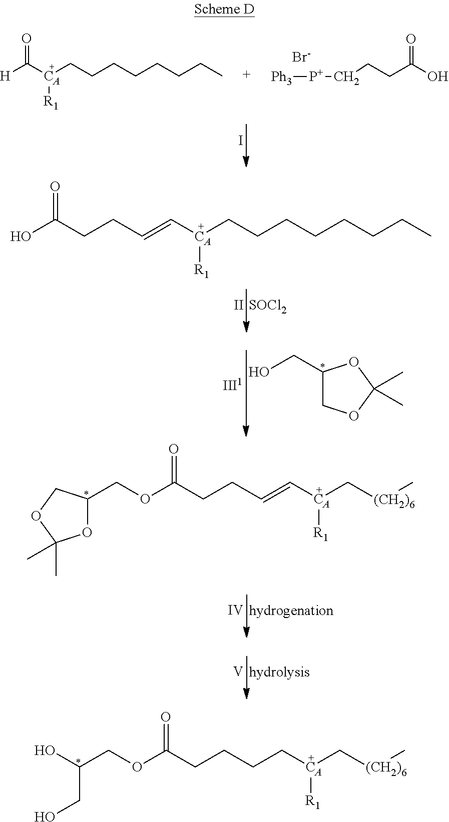

Materials can be selected according to the generalized formulae shown in Scheme D to obtain a compound of the invention having Formula (1) in which A and B are each —OH, V and W are each oxygen, X is —(CH₂)₄— (m=3), Y is —H, and Z is —(CH₂)₆— (n=7). The skilled person would vary reaction conditions for each step to suit the particular materials selected.

The preferred compound of the invention, 6-methyl myristic acid monoglyceride, can also be synthesized according to, for example, Scheme E.

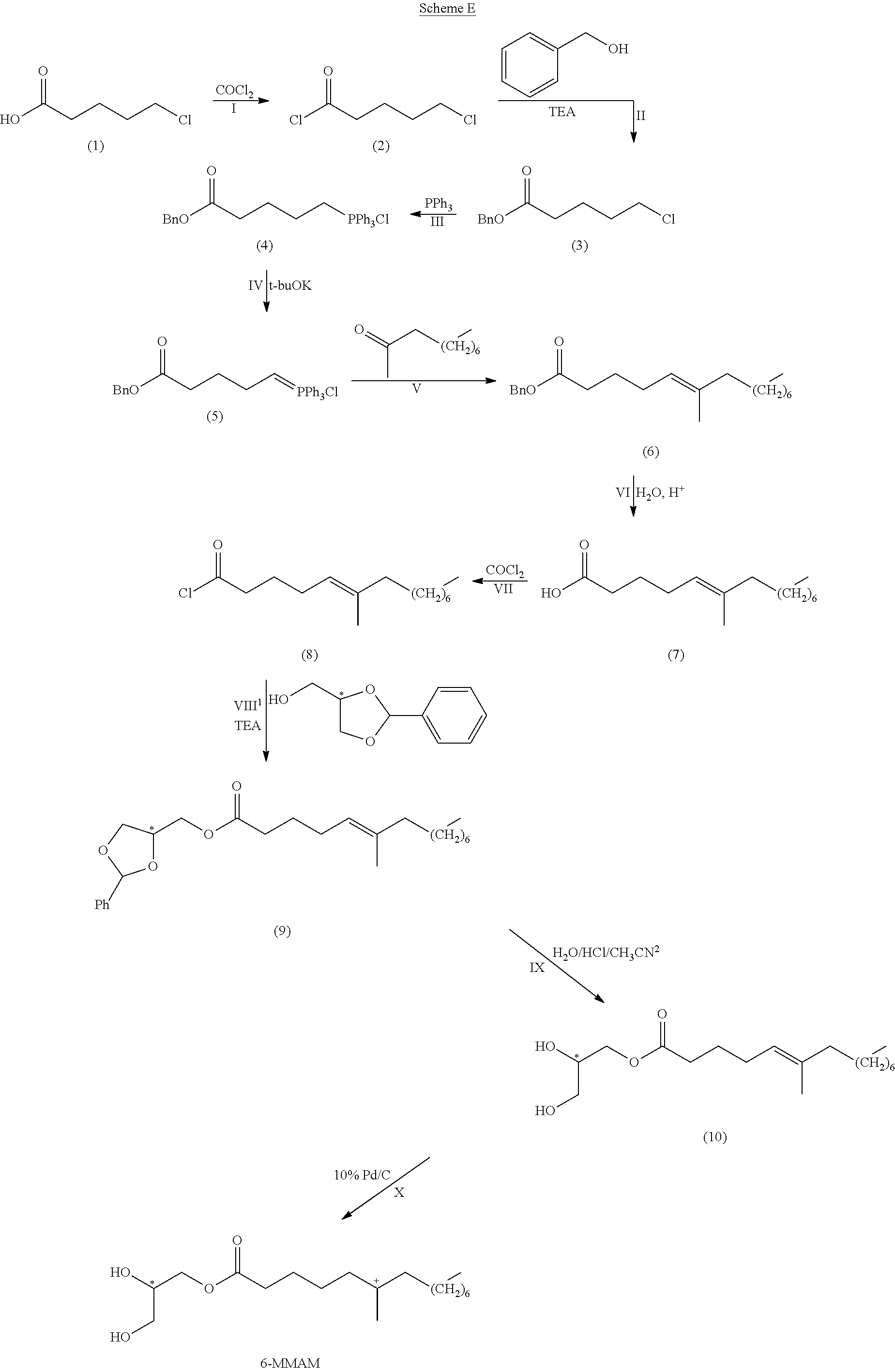

1. The dioxolane of Step VIII can be prepared according to the route shown in Scheme B.
2. R. O. Adlof, W. E. Neff, E. A. Emken, and E. H. Pryde, Journal of the American Oil Chemists' Society, 1977, 54(10):414-416.

The synthetic route shown in Scheme E would produce a mixture of all four stereoisomers, but can be modified to obtain each of the four stereoisomers.

Step VIII of Scheme E can thus be modified as described in connection with Step III of Scheme A, and Scheme C.

The second chiral center (C⁺) in 6-MMAM is introduced by reduction of the double bond in Step X in Scheme E. Compound (6), the product of Step V of Scheme E, would be prepared under conditions in which the longer alkyl chains are formed trans to each other as exemplified in the illustrated Wittig reaction. Asymmetric hydrogenation of the C═C bond will result in formation of either the R— or S— configuration at C-6 of the 6-MMAM. Asymmetric hydrogenation across C═C bonds is well known. See, for example, U.S. Pat. No. 6,878,665 of de Paule et al.

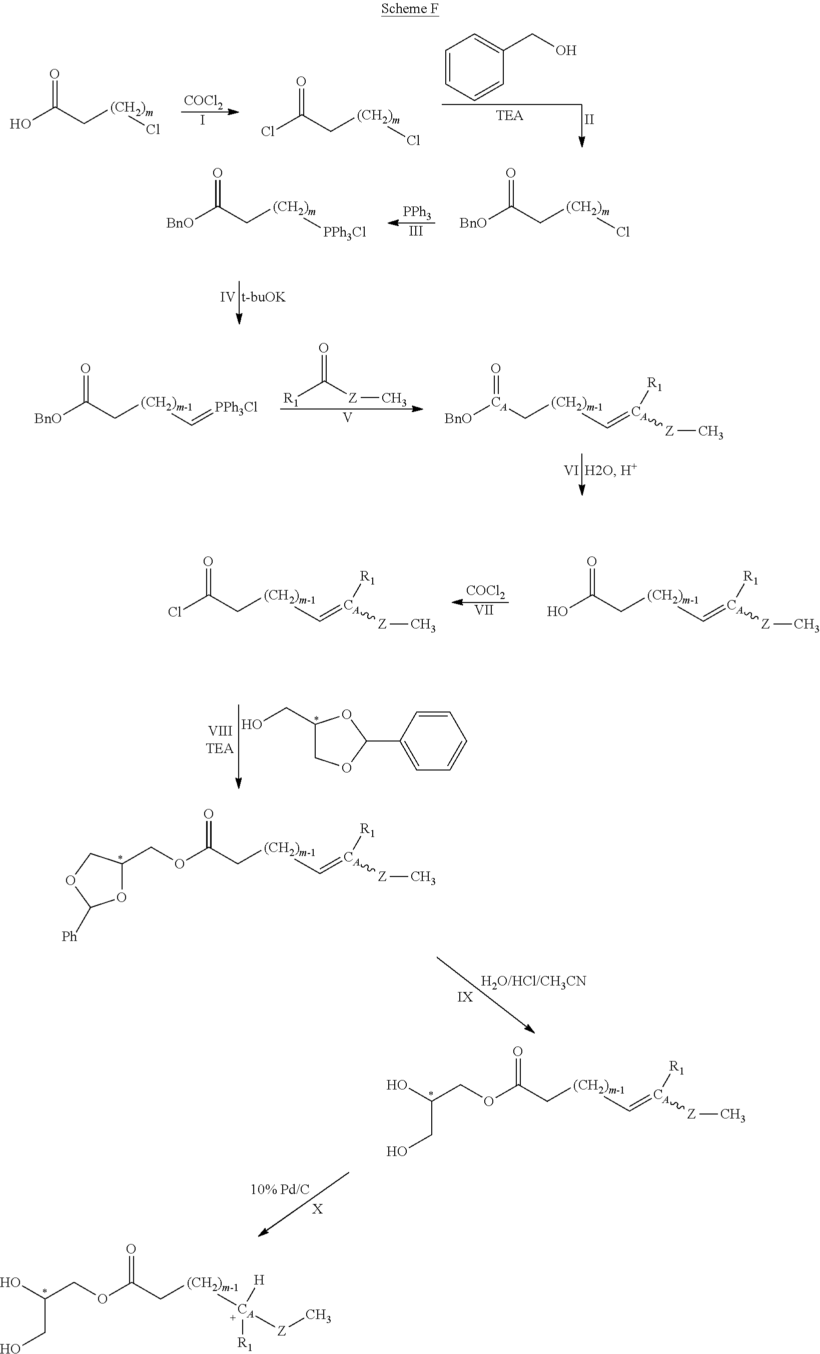

Materials can be selected according to the generalized formulae shown in Scheme F to obtain a compound of the invention having Formula (1) in which A and B are each —OH, V and W are each oxygen, R₁ is —(CH₂)ₚCH₃ or is —H, p is an integer from 0 to 3, X is —(CH₂)ₘ—, Y is —H, Z is —(CH₂)ₙ—, and m and n are integers in which m=1 to 5 and n=4 to 14. The skilled person would vary reaction conditions for each step to suit the particular materials selected.

A stereochemically pure compound of the invention is one in which at least 90% of the compound has the desired stereochemistry e.g., R at C⁺ and S at C*, or R,S at C⁺ and R at C*, etc. More preferably, the compound is at 92% stereochemically pure, more preferably still, 94% stereochemically pure, more preferably still, 96% stereochemically pure, more preferably still, 98% stereochemically pure, and most preferably greater than 99% stereochemically pure. A substantially stereochemically pure compound is one that is at least 96% of the desired optically active stereoisomer(s).

A process for preparing a compound of Formula A6 or A7 is illustrated in Scheme G. The process includes reacting compound A1 and A2 to form alkenyl compound A3. The acid halide of A3 is then formed, and reacted with dioxolane A4 to form A5, which can then be hydrolyzed to form A6, or the C═C double bond of A5 reduced, with subsequent hydrolysis of the dioxolane, to form the compound A7

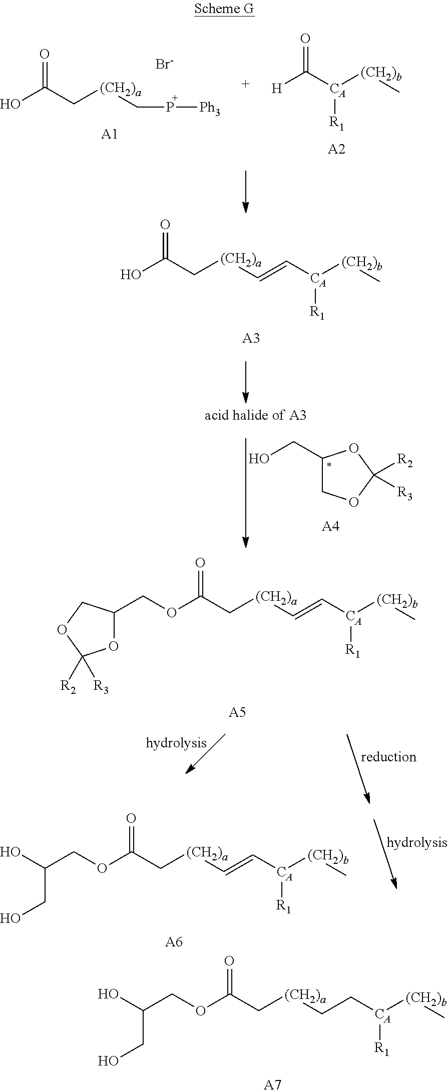

in which: a is an integer from 1 to 3; b is an integer from 1 to 11; 4≦a+b≦12, and R₁ is —(CH₂)ₚCH₃; and p is an integer from 0 to 3. Preferably, p is 0. R₂ and R₃ can be the same or different, and can be any convenient group suitable for the reaction. Specific groups include those disclosed by Hinoue: hydrogen, C₁-C₄ alkyl, or phenyl.

The R or S stereoisomer of dioxolane A4 can be used to obtain A6 or A7, as desired, that is stereochemically pure at C-2 of the glycerol moiety of A6 or A7.

### Functional Analogs of the Compound of Formula (I):

Functional analogs of the compound of Formula (I) can be made using methods known to those skilled in the art. For example, the compound of Formula (I) isolated and identified or synthesized according to the methods described above may be subjected to directed or random chemical modifications, such as replacement of hydrogen by halogen, replacement of an alkyl by a different alkyl, replacement of alkoxy by alkyl, replacement of alkyl by alkoxy, acylation, alkylation, esterification, amidification, etc., to produce structural analogs of the compound, which can be tested for biological activity (e.g., binding to the NK₂ receptor or changes in intracellular calcium concentration as a result of receptor activity) using methods described herein or other methods known to one skilled in the art.

Another way to obtain functional analogs of the compound of Formula (I) is through rational design. This is achieved through structural information and computer modeling. Prediction of target molecule-compound interaction when small changes are made in one or both can be done using molecular modeling software and computationally intensive computers. Examples of molecular modeling systems are the CHARMm and QUANTA programs, Accelrys Inc., San Diego, Calif. CHARMm performs the energy minimization and molecular dynamics functions. QUANTA performs the construction, graphic modeling and analysis of molecular structure. QUANTA allows interactive construction, modification, visualization, and analysis of the behavior of molecules with each other. Other computer programs that screen and graphically depict chemicals are known to those skilled in the art. Functional analogs obtained through rational drug design can also be tested for biological activity (e.g., binding to the NK₂ receptor or changes in intracellular calcium concentration as a result of receptor activity) using methods described herein or other methods known to one skilled in the art.

### Therapeutic Uses for the Compounds of the Invention:

As described in PCT publication number WO 2009/086634, the entire teachings of which are incorporated herein by reference, fertilized egg isolate can be used to treat patients suffering from mental health disorders, including depressive mood disorders, such as major depressive disorder, dysthymic disorder, depressive phase of bipolar disorder, depression due to a general medical condition such as depression associated with dementia or schizoaffective disorder, substance-induced depression and seasonal affective disorder, anxiety disorders, such as generalized anxiety disorder, social phobia and panic disorder, and sexual dysfunction.

As also described in PCT publication number WO 2009/086634, it has been determined that the fertilized egg isolate as described herein antagonizes the binding interactions of certain ligands with their receptors. In particular, it has been found that the fertilized egg isolate has the capacity to displace the neurotransmitter neurokinin A (NKA) from its receptor, the neurokinin 2 (NK₂) receptor.

A number of diseases and conditions are known to be associated with modulation of the NK₂ receptor. Such diseases or conditions include depressive mood disorders, such as major depressive disorder (see, for example, Dableh, Ahlstedt, Michale, Louis, Steinberg, Salomé, Holmes, Steinberg, Husum), anxiety (see, for example, Ahlstedt, Michale, Louis, Greibel, Steinberg, Stratton, Teixeira, Walsh, Salomé, Holmes), irritable bowel syndrome and inflammatory bowel disease (see, for example, Ahlstedt, Lecci, Evangelista, Toulouse), inflammatory airway disease, such as asthma or chronic pulmonary obstructive disorder (COPD) (see, for example, Bai, Pinto, Khawaja) and urinary incontinence (see, for example, Ahlstedt, Rizzo). Furthermore, it has been shown that antagonists of the NK₂ receptor, such as saredutant (SR 48964) can be used to promote antidepressant-like effects (Salomé, Dableh, Steinberg, Michale, Louis) and anxiolytic effects (Teixeira, Salomé, Griebel, Michale, Louis) in animal models, and studies in humans have also been conducted. The modulation of activation of the NK₂ receptor, for example, by inhibiting NK₂'s endogenous ligand(s) (e.g., NKA) from binding to its receptor, can diminish or eliminate disorders or diseases associated with NK₂ receptor activity.

A compound having the structure depicted by Formula (I) has been isolated from the fertilized egg isolate as described herein and has the capacity to displace the neurotransmitter neurokinin A (NKA) from its human NK₂ receptor. The compound of Formula (I) has also been synthesized and found to displace the neurotransmitter neurokinin A (NKA) from the human NK₂ receptor and alter downstream intracellular calcium levels. Accordingly, a compound of Formula (1), Formula (I), as well as functional analogs and pharmaceutically acceptable salts thereof can be used to treat disorders or diseases associated with NK₂ receptor activity.

Accordingly, another aspect of the invention features a method for treating a disorder or disease associated with NK₂ receptor activity using a compound of Formula (1), Formula (I) or a functional analog or pharmaceutically acceptable salt thereof, the method comprising the step of administering a therapeutically effective amount of the compound of Formula (1), Formula (I) or a functional analog or pharmaceutically acceptable salt thereof to a patient in need thereof. The disorder or disease associated with NK₂ receptor activity can be, for example, a depressive mood disorder, such as major depressive disorder, anxiety, inflammatory bowel disease, irritable bowel syndrome, inflammatory airway disease or urinary incontinence.

As will be appreciated by one skilled in the art, a compound of Formula (1), Formula (I) or a functional analog or pharmaceutically acceptable salt thereof can be used to treat disorders or conditions with which depression is associated, such as disorders of the brain or nervous system, substance abuse, eating disorders and hormone disorders, such as thyroid dysfunction, hypogonadism, menopause, etc. In addition, such a compound or analog or pharmaceutically acceptable salt thereof can be used to treat other conditions for which antidepressants have been demonstrated to be effective, such as hot flashes associated with menopause, pain and smoking cessation.

In the methods for treating a disorder or disease or condition associated with NK₂ receptor activity, the patient may or may not be being treated by psychotherapy concurrently with the treatment.

The compounds of the present invention can be formulated for and administered as various dosage forms, such as those adapted for administration by the oral (including buccal, sublingual and by oral inhalation), nasal, topical (including buccal, sublingual and transdermal), or parenteral (including subcutaneous, intramuscular, intravenous or intradermal) routes. Particularly preferred are dosage forms adapted for administration by the oral route. Other preferred dosage forms include those adapted for administration by the vaginal or rectal route, such as a suppository.

### Pharmaceutical Compositions:

The present invention provides compositions for the treatment of a disorder or disease associated with NK₂ receptor activity, such as a depressive mood disorder (e.g., major depressive disorder), anxiety disorder, irritable bowel syndrome, inflammatory bowel disease, inflammatory airway disease or urinary incontinence. In one embodiment, the composition comprises one or more compounds of the invention, or a pharmaceutically acceptable salt thereof. In another embodiment, a composition of the invention comprises one or more compounds of the invention, or a pharmaceutically acceptable salt thereof, and one or more other prophylactic or therapeutic agents. In another embodiment, the composition comprises a compound of the invention, or a pharmaceutically acceptable salt thereof, and a pharmaceutically acceptable carrier, diluent or excipient. In another embodiment, the composition is formulated such that it crosses the blood brain barrier.

A composition of the present invention can be a pharmaceutical composition or a single unit dosage form. Pharmaceutical compositions and dosage forms of the invention comprise one or more active ingredients in relative amounts and formulated in such a way that a given pharmaceutical composition or dosage form can be used to treat a disorder or disease associated with NK₂ receptor activity. Preferred pharmaceutical compositions and dosage forms comprise a compound of Formula (1), Formula (I) or a functional analog or pharmaceutically acceptable salt thereof, optionally in combination with one or more additional active agents.

Single unit dosage forms of the invention are suitable for oral, mucosal (e.g., nasal, sublingual, vaginal, buccal or rectal), parenteral (e.g., subcutaneous, intravenous, bolus injection, intramuscular or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions and elixirs.

The composition, shape, and type of dosage forms of the invention will typically vary depending on their use. For example, a dosage form suitable for mucosal administration may contain a smaller amount of active ingredient(s) than an oral dosage form used to treat the same indication. This aspect of the invention will be readily apparent to those skilled in the art. See, e.g., Remington's Pharmaceutical Sciences (1990) 18th ed., Mack Publishing, Easton, Pa.

Typical pharmaceutical compositions and dosage forms comprise one or more excipients. Suitable excipients are well known to those skilled in the art of pharmacy and/or formulation chemistry, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms.

The invention further encompasses pharmaceutical compositions and dosage forms that comprise one or more compounds that reduce the rate by which an active ingredient will decompose. Such compounds, which are referred to herein as “stabilizers” include, but are not limited to, antioxidants such as ascorbic acid, pH buffers or salt buffers.

### Oral Dosage Forms:

Pharmaceutical compositions of the invention that are suitable for oral administration can be presented as discrete dosage forms, such as, but not limited to, tablets (e.g., chewable tablets), caplets, capsules and liquids (e.g., flavored syrups). Such dosage forms contain predetermined amounts of active ingredients, and may be prepared by methods of pharmacy well known to those skilled in the art. See generally, Remington's Pharmaceutical Sciences (1990) 18th ed., Mack Publishing, Easton, Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives and coloring agents. Examples of excipients suitable for use in solid oral dosage forms (e.g., powders, tablets, capsules and caplets) include, but are not limited to, starches, sugars, micro-crystalline cellulose, diluents, granulating agents, lubricants, binders and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil or soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Piano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Controlled Release Dosage Forms:

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, the entire teaching of each of which are incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with a compound of the invention. The invention thus encompasses single unit dosage forms suitable for oral administration such as, but not limited to, tablets, capsules, gelcaps and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency and increased patient compliance.

Most controlled-release formulations are designed to initially release an amount of drug (active ingredient) that promptly produces the desired therapeutic effect, and gradually and continually releases other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms:

Parenteral dosage forms can be administered to patients by various routes including, but not limited to, subcutaneous, intravenous (including bolus injection), intramuscular and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate and benzyl benzoate.

Compounds that increase the solubility of one or more of the active ingredients disclosed herein can also be incorporated into the parenteral dosage forms of the invention.

### Transdermal, Topical and Mucosal Dosage Forms:

Transdermal, topical and mucosal dosage forms of the invention include, but are not limited to, ophthalmic solutions, sprays, aerosols, creams, lotions, ointments, gels, solutions, emulsions, suspensions or other forms known to one of skill in the art. See, e.g., Remington's Pharmaceutical Sciences (1980 & 1990) 16th and 18th eds., Mack Publishing, Easton, Pa. and Introduction to Pharmaceutical Dosage Forms (1985) 4th ed., Lea & Febiger, Philadelphia, Pa. Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences (1980 & 1990) 16th and 18th eds., Mack Publishing, Easton, Pa.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Dosage & Frequency of Administration:

The amount of a compound or composition of the invention which will be effective in the treatment of a disorder or disease associated with NK₂ receptor activity, or one or more symptoms thereof, will vary with the nature and severity of the disorder or disease, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (62nd ed., 2008).

In general, the recommended daily dose range of a compound of the invention for the disorders or diseases described herein lie within the range of from about 0.01 mg to about 2000 mg per day, given as a single once-a-day dose or as divided doses throughout a day. In one embodiment, the daily dose is administered twice daily in equally divided doses. Preferably, a daily dose range is from about 5 mg to about 1000 mg per day, more specifically, between about 10 mg and about 500 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust or terminate therapy in conjunction with individual patient response.

Prophylactic or therapeutic agents other than compounds of the invention, which have been or are currently being used to treat a disorder or disease associated with NK₂ receptor activity, or one or more symptoms thereof can be used in the combination therapies of the invention. For example, the compounds of the invention can be formulated with other antidepressants such as those that may inhibit the breakdown of serotonin, such as monoamine oxidase inhibitors. In one embodiment, the additional therapeutic agent is one that binds to a glutamate receptor, for example, the AMPA receptor, the kainate receptor, the agonist site of the NMDA receptor or the glycine site that is strychnine-insensitive of the NMDA receptor.

Examples of useful therapeutic agents for treating or preventing depression include, but are not limited to, tricyclic antidepressants such as amitriptyline, amoxapine, bupropion, clomipramine, desipramine, doxepin, imipramine, maprotiline, nefazadone, nortriptyline, protriptyline, trazodone, trimipramine and venlafaxine; selective serotonin reuptake inhibitors such as fluoxetine, fluvoxamine, paroxetine and sertraline; monoamine oxidase inhibitors such as isocarboxazid, pargyline, pheneizine and tranylcypromine; and psychostimulants such as dextroamphetamine and methylphenidate.

Other examples of useful antidepressants include, but are not limited to, binedaline, caroxazone, citalopram, dimethazan, fencamine, indalpine, indeloxazine hydrocholoride, nefopam, nomifensine, oxitriptan, oxypertine, thiazesim, benmoxine, iproclozide, iproniazid, nialamide, octamoxin, phenelzine, cotinine, rolicyprine, rolipram, metralindole, mianserin, mirtazepine, adinazolam, amitriptylinoxide, butriptyline, demexiptiline, dibenzepin, dimetacrine, dothiepin, fluacizine, imipramine N-oxide, iprindole, lofepramine, melitracen, metapramine, noxiptilin, opipramol, pizotyline, propizepine, quinupramine, tianeptine, adrafinil, benactyzine, butacetin, dioxadrol, duloxetine, etoperidone, febarbamate, femoxetine, fenpentadiol, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, L-tryptophan, viloxazine and zimelidine.

Examples of useful therapeutic agents for treating or preventing anxiety disorder include, but are not limited to, benzodiazepines, such as alprazolam, brotizolam, chlordiazepoxide, clobazam, clonazepam, clorazepate, demoxepam, diazepam, estazolam, flumazenil, flurazepam, halazepam, lorazepam, midazolamn, nitrazepam, nordazepam, oxazepam, prazepam, quazepam, temazepam and triazolam; non-benzodiazepine agents, such as buspirone, gepirone, ipsapirone, tiospirone, zolpicone, zolpidem and zaleplon; tranquilizers, such as barbituates, e.g., amobarbital, aprobarbital, butabarbital, butalbital, mephobarbital, methohexital, pentobarbital, phenobarbital, secobarbital and thiopental; and propanediol carbamates, such as meprobamate and tybamate.

Examples of useful therapeutic agents for treating or preventing inflammatory bowel disease include, but are not limited to, anticholinergic drugs, diphenoxylate, loperamide, deodorized opium tincture, codeine; broad-spectrum antibiotics such as metronidazole, sulfasalazine, olsalazine, mesalamine, prednisone, azathioprine, mercaptopurine and methotrexate.

Examples of useful therapeutic agents for treating or preventing irritable bowel syndrome include, but are not limited to, propantheline; muscarine receptor antogonists such as pirenzapine, methoctramine, ipratropium, tiotropium, scopolamine, methscopolamine, homatropine, homatropine methylbromide and methantheline; and antidiarrheal drugs such as diphenoxylate and loperamide.

Examples of useful therapeutic agents for treating or preventing urinary incontinence include, but are not limited to, propantheline, imipramine, hyoscyamine, oxybutynin and dicyclomine.

Examples of useful therapeutic agents for treating or preventing inflammatory airway disease include, but are not limited to, anti-inflammatory agents, such as corticosteroids; leukotriene modifiers; mast cell stabilizers; and bronchodilators such as beta-adrenergic agonists, drugs with anticholinergic effects, and methylxanthines.

Preferably, dosages lower than those which have been or are currently being used to treat a disorder or disease associated with NK₂ receptor activity, or one or more symptoms thereof, are used in the combination therapies of the invention. The recommended dosages of agents currently used for the prevention, treatment, management, or amelioration of a disorder or disease associated with NK₂ receptor activity, or one or more symptoms thereof, can be obtained from any reference in the art including, but not limited to, Hardman et al., eds., 1996, Goodman & Gilman's The Pharmacological Basis Of Basis Of Therapeutics 9.sup.th Ed, McGraw-Hill, New York; Physician's Desk Reference (PDR) 62nd Ed., 2008, Medical Economics Co., Inc., Montvale, N.J., which are incorporated herein by reference in their entirety.

In certain embodiments, when the compounds of the invention are administered in combination with another therapy, the therapies (e.g., prophylactic or therapeutic agents) are administered simultaneously or separately, for example, less than 30 minutes, 1 hour, 3 hours, 5 hours, 10 hours, 12 hours, 18 hours, 24 hours, 36 hours, 48 hours apart, or 72 hours apart.

Another aspect of the invention features a method for modulating an activity of an NK₂ receptor comprising contacting the NK₂ receptor with an effective amount of a compound of the present invention.

The activity of an NK₂ receptor can be modulated by increasing or decreasing (i.e., inhibiting) the activity of the NK₂ receptor. The activity of the NK₂ receptor can be decreased or inhibited, for example, by inhibiting binding of the receptor by its endogenous ligand(s) (e.g., NKA for the NK₂ receptor), or by commercially available exogenous ligands, such as saredutant. Methods for inhibiting such binding interactions and for detecting such binding inhibition are known to those skilled in the art, and are also described herein. Activity of the NK₂ receptor can be decreased by 100% or by less than 100% (for example, by 90%, 80%, 70%, 60%, 50%, 40%, 30%, 20% or 10%). Inhibition of NK₂ receptor activity can occur, for example, by the compound of Formula (1) or Formula (I) binding to the endogenous ligand binding site, thereby decreasing binding by the endogenous ligand. Inhibition of NK₂ receptor activity can also occur but the binding of the compound of Formula (1) or Formula (I) to a site on the NK₂ receptor that is different than the endogenous ligand binding site, yet alters (e.g., decreases) the activity of the NK₂ receptor upon interaction with its endogenous ligand (e.g., allosteric modification of the receptor). Activity of the NK₂ receptor can be increased by 5% or by more than 5% (for example, by 10%, 20%, 30%, 40%, 50%, 60%, 70%, 80% or 90%, 100% or more than 100%). Methods for modulating the activity of an NK₂ receptor can be carried out in vitro (for example, in a cell, cell lysate, or a sample containing a portion of a cell, for example, just the relevant receptor) or in vivo (for example, in a human patient).

### Other Embodiments

The compounds of the invention may be used as research tools (for example, to evaluate the mechanism of action of new drug agents, to isolate new drug discovery targets using affinity chromatography, as antigens in an ELISA or ELISA-like assay, or as standards in in vitro or in vivo assays). These and other uses and embodiments of the compounds and compositions of this invention will be apparent to those of ordinary skill in the art.

The invention is further defined by reference to the following examples describing in detail the preparation of compounds of the invention. It will be apparent to those skilled in the art that many modifications, both to materials and methods, may be practiced without departing from the purpose and interest of this invention. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Example 1

### Preparation of Fertilized Egg Isolate A

To produce fertilized egg Isolate A, 8-9 day old whole fertilized hen eggs were disinfected with 70% ethanol and left in a fume hood to allow the solvent to evaporate. The eggs were then broken and the contents dropped on or through a sterile 1.0 mm mesh. The shells and filtrate were discarded. The retentate, which comprised the embryo, clear sac, and all or a substantial part of the albumen and consisted of solid and semi-solid and/or liquid portions, was chilled on ice and then homogenized at 5° C. The homogenate (slurry) was poured into sterile stainless steel trays, and freeze-dried. The dried product was pulverized in a grinder to give Isolate A. To Isolate A, the preservatives sodium benzoate (0.5% w/w) and potassium sorbate (0.2% w/w) were added and the mixture was blended. The finished powder was stored at 2-8° C. (short term) or −20° C. (long term).

HPLC Analysis

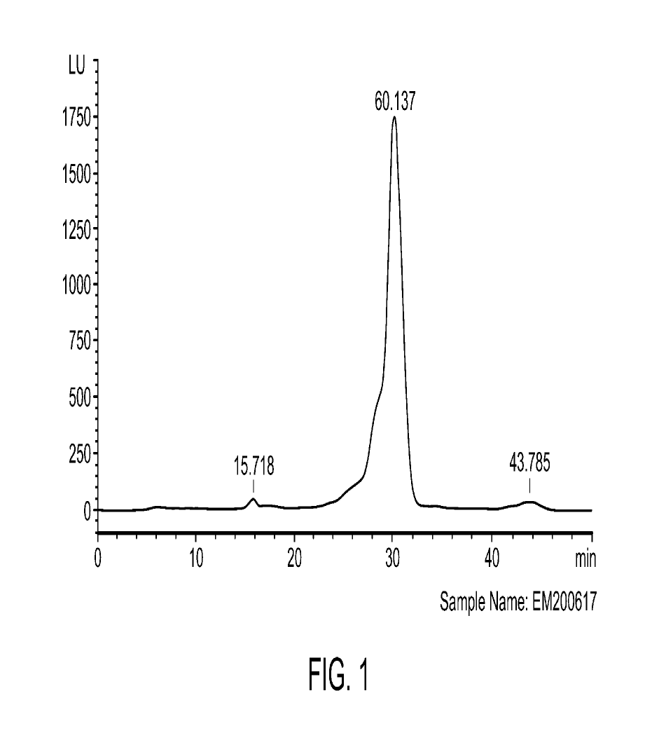

The finished powder containing fertilized egg Isolate A was analyzed by High Performance (or Pressure) Liquid Chromatography (HPLC). The results were quantified using a multiple-wave absorption detector. Absorption was read at 215 nm. A Pharmacia Superdex 200 10/300GL size exclusion column (10 mm i.d.×300 mm) was used for fractionation. The separation range of the column was 10 kDa-600 kDa. The column was equilibrated with 20 mM phosphate+0.3 M NaCl, pH 7.5. The sample was analyzed at a flow rate of 0.5 mL/min. A representative chromatogram is shown at FIG. 1.

Certificate of Analysis

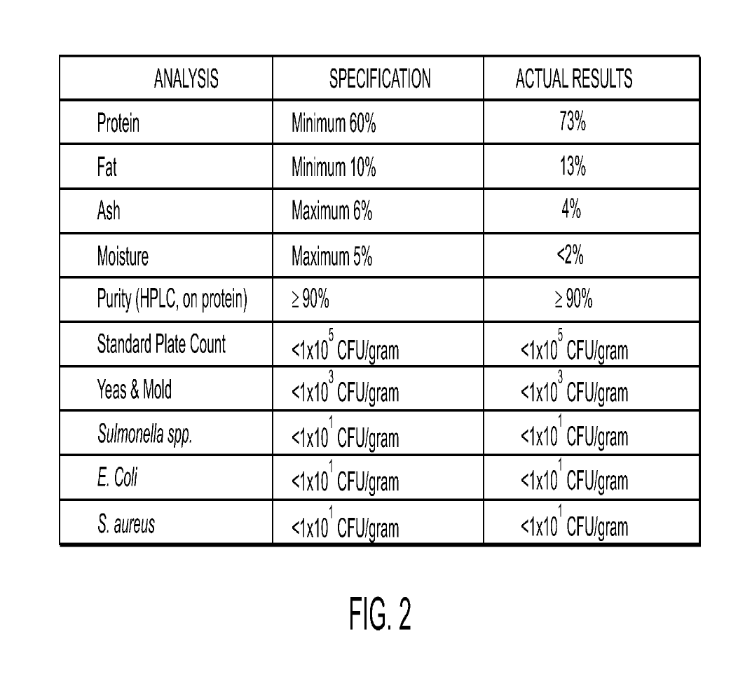

The finished powder containing Isolate A was also subjected to standard analytical procedures to measure purity and the content of protein, fat, ash, moisture, and various contaminants. A representative sample of the results is shown at FIG. 2.

Formulation A Capsules

In order to prepare capsules of Formulation A, 4000.0 g (+/−2%) of the finished powder containing Isolate A, sodium benzoate (0.5% w/w) and potassium sorbate (0.2% w/w) was mixed with 40 g (+/−2%) of fumed silica using geometric dilution. The mixture was sifted, and the mixing and sifting were repeated, resulting in Formulation A. The Formulation A mixture was encapsulated using Mini-Cap 300#0 white capsules to a target fill weight of 505 mg to produce Formulation A capsules.

### Example 2

### Study of Formulation A for Treatment of Major Depressive Disorder (MDD) and Disorders/Symptoms Related Thereto

The efficacy and safety of a fixed dose of Formulation A to treat mental disorders, such as MDD and related disorders and symptoms, were studied. This study included evaluation of the effect of Formulation A on reducing symptoms of anxiety, improving quality of life, and improving symptoms of sexual dysfunction.

Description of Evaluation Techniques

Hamilton Depression Rating Scale-17 Item—“HAM-D” or “HAM-D 17”

This is a leading rating scale used in North America for evaluating depression in a patient. The total scores are interpreted as follows: very severe, >23; severe, 19-22; moderate, 14-18; mild, 8-13; and no depression, 0-7.

Hamilton Anxiety Rating Scale-14 Item—“HAM-A”

This rating scale evaluates the level of anxiety in a patient. The score levels are interpreted as follows: <17, mild; 18-24, mild to moderate; and 25-30, moderate to severe.

Montgomery-Åsberg Depression Rating Scale—“MADRS”

This is a leading rating scale used in North America for evaluating depression in a patient. The following mean scores correlate with global severity measures, according to a study: very severe, 44; severe, 31; moderate, 25; mild, 15; and recovered, 7.

Beck Depression Inventory—“BDI”

This is a commonly employed measure of depressive symptoms typically used as a self-assessment instrument. The total score is the simple sum of the 21 item scores. Generally, a score<9 indicates no or minimal depression, 10-18 indicates mild-to-moderate depression, 19-29 indicates moderate-to-severe depression, and >30 indicates severe depression. However, a score of 0-4 may suggest possible denial of depression and a score of 40-63 may suggest possible exaggeration of depression or a histrionic or borderline personality disorder.

Arizona Sexual Experience Scale—“ASEX”

This is a 5-item rating scale that quantifies sex drive and evaluates levels of arousal, vaginal lubrication/penile erection, ability to reach orgasm, and satisfaction from orgasm.

Possible total scores range from 5 to 30, with the higher scores indicating more sexual dysfunction.

General Health Questionnaire Scoring—“GHQ”

The quality of life dimension may be assessed with the Short-Form 36 (SF-36). This questionnaire evaluates such problems as the ability to concentrate, feelings of worry, low self-confidence, feelings of low self-worth, unhappiness, and depression. The scoring is as follows:

Likert Scale 0, 1, 2, 3 from left to right; 12 items were assessed on a scale of 0 to 3 for each item.

Score Range 0 to 36.

Scores vary by study of population. Scores about 11-12 are typical.

Score>15 evidence of distress.

Score>20 suggests severe problems and psychological stress.

Diagnostic and Statistical Manual of Mental Disorders-IV-Text Revision—“DSM-IV TR”

This is the standard diagnostic manual in North America for mental health professionals that comprehensively classifies mental disorders and provides widely accepted criteria for diagnosing them based on the best empirical evidence available.

The primary effect measured was the repeated analysis of variance with the score on the HAM-D as the outcome variable. Secondary effect measures included the CGI-S and CGI-I, MADRS, SF36, BDI, HAMA and ASEX.

Description of Study

An open-label study was carried out at Mount Sinai Hospital (MSH) in Toronto, Ontario, Canada. Patients were recruited by media advertisement, referral from the MSH outpatient program and from other clinical centers.

This protocol describes an open pilot study to investigate Formulation A's potential antidepressant activity. The goal of the pilot study was to demonstrate that Formulation A has the potential to significantly improve MDD beyond the levels of the known placebo effect well established in other trials and that Formulation A is an acceptable treatment in this patient population. Secondary aims of this pilot study were to evaluate the effect of Formulation A on reducing symptoms of anxiety and improving quality of life.

Each patient was screened for MDD using DSM-IV TR criteria and the HAM-D. Once entered, they were assigned to the open-label Formulation A study for a period of 8 weeks. The patients were further assessed by a global measure, the CGI severity (GCI-S) and improvement (CGI-I) scales. Side effects were systematically evaluated using The Udvalg for Kliniske Undersøgelser (UKU) Side Effect Rating Scale (Lingjaerde). Secondary measures of depressive symptoms were the Montgomery Åsberg Depression Rating Scale (MADRS) and the Beck Depression Inventory (BDI) as self assessment instruments. The quality of life dimension was assessed with the Short-Form 36 (SF-36). Anxiety was assessed using the 14 item HAM-A.

In the fixed dose open trial, patients were treated for depression based on standard treatment protocols for depression. The investigators determined the severity of depression with the rating scales at baseline, and at repeated visits at weeks (W) 2, 4, 6, and at week 8. In intervening weeks patients were seen in brief clinical assessments (V) to evaluate depression and medication tolerance.

Dosage of Formulation A

The dosage of Formulation A was about 2000 mg/day (two Formulation A capsules of about 500 mg each, taken orally twice a day).

Inclusion Criteria of Patients

For inclusion in this study, patients had to have met a number of inclusion criteria, including criteria (i)-(vi), as described below.

(i) A clinical diagnosis fulfilling DSM-IV TR criteria for major depressive disorder, single episode or recurrent.

(ii) 17-Item Hamilton Depression Rating Scale (HAM-D 17-item) total score at baseline of 18 or higher.

(iii) Males/Females 18-65 years of age who require a new or a change in their medication treatment for diagnosed major depression. Treatment decisions were made solely upon the clinician's judgment of the standard of care appropriate to that patient. However augmentation strategies were not permitted during the 8 week trial.

(iv) English language literacy.

(v) Signed written informed consent obtained.

(vi) A negative pregnancy test at screening.

Exclusion Criteria

Patients were excluded from this study if they met a number of exclusion criteria, including criteria (i)-(xiii), as described below.

(i) Any other DSM IV TR diagnosis including a clinical diagnosis of depression other than DSM-IV TR MDD (single episode/recurrent, e.g., chronic depression and/or refractory depression were excluded).

(ii) Judged to be at significant risk for suicide (HAMD suicide item>1) or having a history suggesting significant current potential for self harm.

(iii) Any antidepressant medication other than Formulation A.

(iv) Subjects who were taking and unable or unwilling to discontinue natural health products used for depression.

(v) Women who were pregnant, breast-feeding, intending to become pregnant in the next 12 months or on insufficient contraceptive protection.

(vi) Clinically significant organ system diseases, e.g., cardiovascular, hepatic, renal, endocrine, gastrointestinal, metabolic, or other systemic diseases.

(vii) Course of electroconvulsive therapy (ECT) during the observational period.

(viii) Suffer from a major neurological condition (i.e., Parkinson's disease, Huntington's disease), cerebrovascular disease (i.e., stroke), metabolic conditions (i.e., Vitamin B12 deficiency), autoimmune conditions (i.e., systematic lupus erythematosus), viral or other infections (i.e., hepatitis, mononucleosis, human immunodeficiency), or cancer.

(ix) Clinical or subclinical hypo/hyper thyroidism (e.g., elevated TSH).

(x) Allergies to poultry or eggs.

(xi) Subjects who were receiving psychotherapy or who began psychotherapy during the trial.

(xii) Subjects with clinically significant abnormal laboratory results from screening blood and urinalysis.

(xiii) Subjects who became significantly worse during the washout period.

Study Design

This was a single site, open-label, randomised study of 23 patients (20 of whom had analyzable results) designed to validate the efficacy and safety of Formulation A monotherapy.

The trial consisted of an 8 week evaluation period preceded if necessary by a 2 week antidepressant washout period.

Screening

Once the physician and/or research coordinator fully informed the subject of the study, the nature of the treatment, and the other options available to them, and the subject signed the informed consent document, the physician made the clinical DSM IV TR diagnosis and administered the HAM-D 17. Eligible subjects then had a medical, psychiatric history and concomitant therapy review followed by a physical examination. In addition, baseline laboratory tests were taken by the research coordinator including urine (Routine & Microscopic), CBC differential and platelets, electrolytes, bilirubin, BUN, creatinine, TSH, Liver Function Tests, Serum creatinine, and ECG. A pregnancy screen for female patients was obtained by hCG blood test. Pregnant patients and those with clinically significant abnormal laboratory tests were excluded.

Week 0

Patients returned for a Baseline visit (Week 0) and were assigned to Formulation A monotherapy by the physician. Patients who were depressed and on a current but ineffective antidepressant were offered the switch to Formulation A.

Following Weeks

Following the initial assessment and initiation of Formulation A therapy (V1 and V2) the scheduled visits occurred every week for 8 weeks (W2-W8, V3-V6). Those who were on another antidepressant drug and who chose to enter the study entered a 1-2 week washout period before beginning the 8 week active drug trial. The washout period was at the clinical discretion of the physician. During this time, patients were monitored in a visit one week into the washout by the psychiatrist and further monitored by phone by the study coordinator mid-week. It is recognized that depression may worsen during the washout period. However, if the prior drug was ineffective or partially ineffective the risks that a 1-2 week delay will significantly induce depressive decline in this protocol are not substantially greater than usual care as long as subjects are carefully monitored during this time and appropriate intervention instituted as necessary. If Formulation A were not to be an effective antidepressant for a particular patient, the patient may be at risk for undue prolongation of depression. However, depression is a chronic disorder which is generally present for months prior to being diagnosed or treated so an additional 8 weeks in the presence of careful monitoring together with institution of Formulation A, a potentially effective medication, should not be substantially different from standard care. Moreover, standard care, as already discussed, is only effective in about 60% of patients and therefore often requires the same possible reevaluations and drug alterations.

At V2 (may be combined with V1 (WO)) to V6 (W8), the following procedures were performed by the supervising psychiatrist (PI) and/or the research coordinator:
Weight
Height
Vital Signs
Hamilton Depression Rating Scale (17 item) (HAM-D 17) (Hamilton 1967).
Clinical Global Impression (CGI-S, CGI-I) (Guy)
Montgomery-Åsberg Depression Rating Scale (MADRS) (Montgomery)
Beck Depression Inventory Scale (BDI) (10). —Quality of Life (SF-36) (Ware).
Hamilton rating scale for anxiety (HAMA) (Hamilton 1959)
Udvalg for Kliniske Undersogelser (UKU) (Lingjaerde) (Reporting of Adverse Events) (except at V2)
Medication Compliance (except at V2)

Study visits were estimated at about one hour with the exception of the baseline visit which may have taken 2 hours.

If subjects became more depressed while in the study they were evaluated by the principal investigator to determine the best clinical approach. If deemed necessary, Formulation A was stopped in favour of another antidepressant treatment. This was a clinical decision made solely on the basis of best practices in the treatment of depression and on the patient's best clinical interests.

General supportive contact with the patients by the physician and the research coordinator was permitted, and the contact was generally restricted to answering pertinent questions about the patient's illness course and treatment. No formal psychotherapy was permitted.

Statistical Methods

The primary effect was tested using a repeated analysis of variance with the scores of the HAM-D 17 as the outcome variable. A significant time effect supports the hypothesis. The total anticipated sample size of 25 patients was large enough to detect changes on the HAM-D 17 as follows 0.65 standard deviations (two-tailed one sample P<0.05). The reported standard deviations on the HAM-D-17 were in the range of 4.5 to 6.5. Therefore, the design of this study had 80% power to detect average changes as small as 4.3 points on this 52 point scale. As per inclusion criteria, participants each had a HAM-D 17 score of greater than 17. The Franck criterion for remission was a HAM-D 17 of 9 or under. This study used a more conservative and accepted level of 7 or under. The effect size of 4.3 was sufficiently sensitive to detect clinical improvement from scores greater than 17 to scores less than 10. Positive outcome was statistically based on an expected placebo response rate ranging from 30% to 50% in treatment trials for depression. In this study, a placebo response rate of 40% was assumed. Some analysis of responders and remitters was carried out as appropriate.

Results

A total of 23 patients were entered into the study. Three of the subjects, (#104, #105 and #118) were never treated and hence, their results were not considered analyzable. Of the subjects that received at least one dose of Formulation A, 16 of them completed the 8 week study. The remaining 4 subjects did not complete the 8 week study, but since they each received at least one dose of Formulation A, their results were deemed analyzable. The reasons these 4 subjects did not complete the entire study included non-compliance with the medications and/or appointments, impatience with the results, and the subject leaving the country.

The results for the 20 subjects who received at least one dose of Formulation A are provided in the below tables.

### Total Score Sheet for HAM-D

| Screen | Week 0 | Week 2 | Week 4 | Visit 7 | Week 6 | Week 8 |
|---|---|---|---|---|---|---|
| Subject #101 | 20 | 14 | | 11 | 7 | 3 |
| Subject # 102 | 19 | 15 | 5 | | 2 | 0 |
| Subject # 103 | 22 | 7 | 3 | | 5 | 0 |
| Subject # 106 | 21 | 4 | 8 | | 10 | 12 |
| Subject # 107 | 22 | 17 | 20 | | | |
| Subject # 108 | 20 | 14 | 19 | | | |
| Subject # 109 | 25 | 16 | 17 | | 20 | 24 |
| Subject # 110 | 21 | 10 | 17 | | 8 | 4 |
| Subject # 111 | 24 | 20 | 19 | 19 | 23 | 23 |
| Subject # 112 | 29 | 8 | 5 | | 2 | 0 |
| Subject # 113 | 33 | 13 | 9 | | 11 | 8 |
| Subject # 114 | 29 | 13 | 19 | | 22 | 30 |
| Subject # 115 | 32 | 8 | 13 | | 5 | 6 |
| Subject # 116 | 19 | 17 | 24 | | 17 | 24 |
| Subject # 117 | 23 | 11 | 9 | | 8 | |
| Subject # 119 | 23 | 23 | 20 | | 13 | 10 |
| Subject # 120 | 23 | 5 | | | | |
| Subject # 121 | 23 | 11 | 8 | | 6 | 3 |
| Subject # 122 | 32 | 22 | 16 | | 23 | 16 |
| Subject # 123 | 24 | 19 | 12 | | 10 | 11 |

### Total Score Sheet for GHQ

| Screen | Week 0 | Week 2 | Week 4 | Visit 7 | Week 6 | Week 8 |
|---|---|---|---|---|---|---|
| Subject #101 | 15 | 27 | | 14 | 11 | 3 |
| Subject # 102 | 22 | 11 | 3 | | 0 | 0 |
| Subject # 103 | 18 | 9 | 13 | | 2 | 2 |
| Subject # 106 | 22 | 10 | 10 | | 6 | 10 |
| Subject # 107 | 27 | 12 | 9 | | | |
| Subject # 108 | 27 | 19 | 17 | | | |
| Subject # 109 | 25 | 16 | 16 | | 17 | 20 |
| Subject # 110 | 28 | 13 | 16 | | 9 | 8 |
| Subject # 111 | 26 | 15 | 20 | 21 | 21 | 20 |
| Subject # 112 | 30 | 15 | 9 | | 4 | 0 |
| Subject # 113 | 34 | 13 | 12 | | 5 | 2 |
| Subject # 114 | 33 | 25 | 22 | | 22 | 28 |
| Subject # 115 | 31 | 2 | 7 | | 5 | 8 |
| Subject # 116 | 32 | 26 | 25 | | 25 | 24 |
| Subject # 117 | 24 | 14 | 7 | | 8 | |
| Subject # 119 | 31 | 19 | 27 | | 20 | 8 |
| Subject # 120 | 23 | 8 | | | | |
| Subject # 121 | 35 | 7 | 1 | | 1 | 1 |
| Subject # 122 | 31 | 23 | 10 | | 23 | 15 |
| Subject # 123 | 26 | 17 | 11 | | 4 | 2 |

### Total Score Sheet for MADRS

| Screen | Week 0 | Week 2 | Week 4 | Visit 7 | Week 6 | Week 8 |
|---|---|---|---|---|---|---|
| Subject #101 | 34 | 24 | | 28 | 10 | 6 |
| Subject # 102 | 30 | 18 | 10 | | 0 | 2 |
| Subject # 103 | 28 | 10 | 4 | | 2 | 2 |
| Subject # 106 | 30 | 14 | 14 | | 18 | 24 |
| Subject # 107 | 38 | 28 | 28 | | | |
| Subject # 108 | 20 | 18 | 22 | | | |
| Subject # 109 | 28 | 23 | 20 | | 28 | 26 |
| Subject # 110 | 28 | 16 | 36 | | 14 | 10 |
| Subject # 111 | 46 | 40 | 40 | 40 | 32 | 36 |
| Subject # 112 | 38 | 16 | 10 | | 10 | 2 |
| Subject # 113 | 46 | 16 | 18 | | 14 | 6 |
| Subject # 114 | 42 | 26 | 38 | | 38 | 44 |
| Subject # 115 | 32 | 12 | 12 | | 10 | 10 |
| Subject # 116 | 36 | 42 | 44 | | 34 | 46 |
| Subject # 117 | 36 | 22 | 10 | | 6 | |
| Subject # 119 | 38 | 34 | 34 | | 18 | 10 |
| Subject # 120 | 32 | 6 | | | | |
| Subject # 121 | 38 | 14 | 6 | | 6 | 4 |
| Subject # 122 | 44 | 38 | 22 | | 32 | 24 |
| Subject # 123 | 30 | 28 | 22 | | 14 | 16 |

### Total Score Sheet for BDI-21

| Screen | Week 0 | Week 2 | Week 4 | Visit 7 | Week 6 | Week 8 |
|---|---|---|---|---|---|---|
| Subject #101 | 27 | 25 | | 27 | 17 | 10 |
| Subject # 102 | 25 | 13 | 6 | | 0 | 1 |
| Subject # 103 | 26 | 14 | 10 | | 8 | 8 |
| Subject # 106 | 30 | 12 | 7 | | 12 | 40 |
| Subject # 107 | 33 | 28 | 26 | | | |
| Subject # 108 | 32 | 14 | 20 | | | |
| Subject # 109 | 29 | 23 | 24 | | 20 | 25 |
| Subject # 110 | 29 | 22 | 24 | | 13 | 8 |
| Subject # 111 | 32 | 28 | 27 | 33 | 27 | 27 |
| Subject # 112 | 37 | 21 | 10 | | 9 | 1 |
| Subject # 113 | 53 | 23 | 22 | | 18 | 3 |
| Subject # 114 | 54 | 40 | 52 | | 52 | 59 |
| Subject # 115 | 39 | 13 | 16 | | 3 | 4 |
| Subject # 116 | 38 | 37 | 37 | | 42 | 40 |
| Subject # 117 | 24 | 20 | 11 | | 7 | |
| Subject # 119 | 35 | 36 | 40 | | 24 | 18 |
| Subject # 120 | 26 | 1 | | | | |
| Subject # 121 | 43 | 10 | 4 | | 3 | 3 |
| Subject # 122 | 55 | 38 | 25 | | 46 | 27 |
| Subject # 123 | 33 | 34 | 22 | | 5 | 10 |

### Total Score Sheet for HAM-A

| Screen | Week 0 | Week 2 | Week 4 | Visit 7 | Week 6 | Week 8 |
|---|---|---|---|---|---|---|
| Subject #101 | 15 | 9 | | 8 | 7 | 3 |
| Subject # 102 | 13 | 8 | 2 | | 0 | 1 |
| Subject # 103 | 8 | 7 | 2 | | 0 | 2 |
| Subject # 106 | 21 | 5 | 5 | | 5 | 10 |
| Subject # 107 | 12 | 7 | 13 | | | |
| Subject # 108 | 15 | 8 | 11 | | | |
| Subject # 109 | 17 | 12 | 13 | | 18 | 14 |
| Subject # 110 | 14 | 8 | 9 | | 2 | 5 |
| Subject # 111 | 33 | 29 | 24 | 32 | 23 | 24 |
| Subject # 112 | 22 | 10 | 4 | | 1 | 2 |
| Subject # 113 | 35 | 22 | 14 | | 4 | 3 |
| Subject # 114 | 24 | 10 | 18 | | 21 | 21 |
| Subject # 115 | 25 | 10 | 12 | | 4 | 5 |
| Subject # 116 | 12 | 13 | 15 | | 12 | 16 |
| Subject # 117 | 21 | 10 | 8 | | 7 | |
| Subject # 119 | 13 | 19 | 15 | | 13 | 6 |
| Subject # 120 | 14 | 4 | | | | |
| Subject # 121 | 24 | 9 | 3 | | 4 | 2 |
| Subject # 122 | 43 | 31 | 21 | | 32 | 22 |
| Subject # 123 | 23 | 27 | 14 | | 8 | 10 |

### Total Score Sheet for ASEX

| Screen | Week 0 | Week 2 | Week 4 | Visit 7 | Week 6 | Week 8 |
|---|---|---|---|---|---|---|
| Subject #101 | 12 | 14 | | 12 | 13 | 13 |
| Subject # 102 | 21 | 23 | 19 | | 19 | 15 |
| Subject # 103 | 12 | 12 | 17 | | 17 | 13 |
| Subject # 106 | 10 | 9 | 7 | | 7 | 10 |
| Subject # 107 | 15 | 14 | 16 | | | |
| Subject # 108 | 15 | 11 | 12 | | | |
| Subject # 109 | 17 | 15 | 17 | | 19 | 19 |
| Subject # 110 | 26 | 25 | 28 | | 28 | 27 |
| Subject # 111 | 28 | 28 | 28 | 28 | 28 | 28 |
| Subject # 112 | 11 | 13 | 11 | | 11 | 9 |
| Subject # 113 | 28 | 28 | 28 | | 28 | 9 |
| Subject # 114 | 28 | 28 | 28 | | 30 | 30 |
| Subject # 115 | 19 | 17 | 17 | | 11 | 15 |
| Subject # 116 | 11 | 10 | 11 | | 12 | 12 |
| Subject # 117 | 14 | 12 | 18 | | 16 | |
| Subject # 119 | 30 | 30 | 28 | | 30 | 30 |
| Subject # 120 | 19 | 15 | | | | |
| Subject # 121 | 18 | 26 | 12 | | 22 | 10 |
| Subject # 122 | 26 | 14 | 15 | | 30 | 12 |
| Subject # 123 | 22 | 20 | 20 | | 16 | 20 |

Response Rate and Intensity of Response

The following definitions were used to assess each subject's response to treatment with Formulation A. A “responder” or “ever-responder” is a subject with at least 50% improvement on the Hamilton Depression Rating Scale (HAM-D score) as compared to baseline score at any time during the study. A “clinical responder” is a subject meeting the “responder” criteria who, in the opinion of the Principal Investigator has a positive clinical outcome. An “end of study responder” is a subject meeting response criteria at the end of the study (or at last observation). “Remission” is a reduction of the HAM-D score to less than 8.

The above study showed that of the 20 subjects who received at least one dose of Formulations A, 15 of them (75%) were ever-responders, and 14 of them (70%) were clinical responders. In addition, among the 16 subjects who completed the 8 week study, the number of ever-responders was 13/16 (81.3%) and the number of clinical responders was 12/16 (75%). In addition, of the 16 subjects who completed the study, the overall drop in HAM-D score (including non-responders) was significant at 56.08%. The drop in HAM-D score among the ever-responders who completed the 8 week study was higher, at 68.1%, a figure well beyond the minimal 50% drop rate require for an ever-response.

Note should be made of two subjects whose response was influenced by environmental circumstances. Subject #114, who was not included among the clinical responders, was responsive by week 2 when her HAM-D score fell by more than 50% on Formulation A; but external factors intervened. She began to encounter medical problems (not related to Formulation A) and difficulty at work when she applied for disability insurance. These environmental factors completely overtook her good emotional response to Formulation A.

Based on the strict criterion of a 50% reduction in the HAM-D score, subject #106 would not have been considered a responder at week 8 because at that time her score was 12, just short of a 50% drop from her entry score of 21. Throughout the 8 week trial, however, subject #106 did respond with scores of 4 (week 2), 8 (week 4) and 10 (week 6) In fact, during the study, subject #106 was deemed to be a clinical responder by the PI and was entered into the Extension Study (see Example 3) where scores of 1, 11, 7 and 9 were recorded. After starting the Extension Study, subject #106 was faced with considerable family turmoil which disrupted her positive response to Formulation A. When this turmoil subsided she continued to maintain responsiveness to Formulation A. No medication can completely offset the traumatic effects of environmental circumstances. Formulation A may well have ameliorated the emotional trauma of those circumstances for subject #106.

Remission Rate

Not all ever-responders went into remission and not everyone who went into remission remained there until the end of the 8 week study. Nine of the 15 ever-responders (60%) went into remission at some point during the 8 week study. Seven of those nine subjects (77.8%; or 46.7% of all study participants) who achieved remission remained in remission by the end of the 8 week study.

The table below delineates all those study participants who went into remission and all who stayed in remission. Checkmarks indicate that the subject went into remission or had sustained remission, while X marks indicate that the subject did not go into remission or did not have sustained remission to week 8 of the study.

| | Remission at any | Remission Sustained to 8 |
|---|---|---|
| Subject | time | Weeks |
| 101 | ✓ | ✓ |
| 102 | ✓ | ✓ |
| 103 | ✓ | ✓ |
| 106 | ✓ | X |
| 110 | ✓ | ✓ |
| 112 | ✓ | ✓ |
| 113 | X | X |
| 114 | X | X |
| 115 | ✓ | ✓ |
| 117 | X | X |
| 119 | X | X |
| 120 | ✓ | withdrew |
| 121 | ✓ | ✓ |
| 122 | X | X |
| 123 | X | X |
| | N ever in | N in sustained |
| | remission = 9 | remission = 7 |
| | (60%) | (46.7%) |

In addition, a major secondary outcome, reduction in anxiety, was experienced by all of the ever-responders except one. These results show that Formulation A is effective in the treatment of major depressive disorder and anxiety. Furthermore, there were no serious side-effects attributable to the drug. There was no increase in weight, nor was there a diminution in sexual function in subjects participating in the study.

### Example 3

The positive efficacy and safety results of the study described in Example 2 necessitated an Extension Study. Ten subjects from the study described in Example 2 were entered into the Extension Study. The Extension Study was open only to those subjects from the study described in Example 2 who were clinical responders at the end of that 8 week study. Formulation A was administered as described in Example 1 and the subjects in the Extension Study were analyzed on a monthly basis for 10 months. The below table show the HAM-D scores of the subjects in the Extension Study.

| | Subject | Subject | Subject | Subject | Subject | Subject | Subject | Subject | Subject | Subject |
|---|---|---|---|---|---|---|---|---|---|---|
| Month | # 102 | # 103 | # 106 | # 110 | # 112 | # 113 | # 115 | # 119 | # 121 | # 123 |
| Visit 1 | 0 | 0 | 12 | 4 | 0 | 8 | 6 | 10 | 3 | 11 |
| Visit 2 | 0 | 2 | 1 | 5 | 2 | 9 | 2 | 10 | 4 | 14 |
| Visit 4 | 1 | 8 | 11 | 3 | 1 | 10 | w/d | 3 | 5 | 14 |
| Visit 4 | 0 | 2 | 7 | 1 | 1 | 18 | | 10 | 1 | 3 |
| Visit 5 | 0 | 3 | 9 | 2 | 0 | w/d | | 4 | 5 | |
| Visit 6 | 0 | 1 | 16 | 2 | 1 | | | w/d | | |
| Visit 7 | 0 | w/d | 9 | 2 | 0 | | | | | |
| Visit 8 | 1 | | 6 | 4 | 0 | | | | | |
| Visit 9 | 1 | | 11 | 1 | | | | | | |
| Visit 10 | 2 | | | | | | | | | |

w/d = withdrawn from Extension Study

Four of the 10 subjects were withdrawn from the Extension Study due to occurrence of an exclusionary criterion for continuing in the study. The results of this Extension Study showed that all of the subjects in the study were, by definition, responders to Formulation A. Six of the 10 clinical responders (60%) were in remission at the outset of the Extension Study. Eight of the 10 subjects (80%) were in remission at the last date of assessment. Two of the subjects were clinical responders in the initial 8 week study but had not gone into remission until in the Extension Study. Only one subject (#113) who entered the Extension Study as a clinical responder relapsed after entering the Extension Study.

### Example 4

The positive efficacy and safety results of the Extension Study described in Example 3 necessitated a Second Extension Study. Four subjects from the Extension Study described in Example 3 were entered into the Second Extension Study. The Second Extension Study was open to those subjects from the Extension Study who wanted to continue taking Formulation A. Formulation A was administered as described in Examples 2 and 3. The Second Extension Study is scheduled to last for 12 month, and the four subjects enrolled in the study have currently completed either 8 or 9 months of the study. Each subject has been, and will be, analyzed on a monthly basis. The below table show the HAM-D scores of the subjects in the Extension Study.

| | Subject | Subject | Subject | Subject |
|---|---|---|---|---|
| Month | # 102 | # 106 | # 110 | # 123 |
| Visit 1 | 3 | 1 | 3 | 4 |
| Visit 2 | 0 | 3 | 0 | 2 |
| Visit 4 | 0 | 4 | 1 | 5 |
| Visit 4 | 0 | 7 | 0 | |
| Visit 5 | 0 | 12 | 1 | |
| Visit 6 | 1 | 2 | 2 | |
| Visit 7 | 0 | 2 | 0 | |
| Visit 8 | 0 | | 0 | |
| Visit 9 | 2 | | | |
| Visit 10 | | | | |
| Visit 11 | | | | |
| Visit 12 | | | | |

The results of this Second Extension Study show that all of the subjects in the study (with the exception of subject #106 at visit 5) have remained in remission (i.e., having a HAM-D score of less than 8) throughout the length of time they have been enrolled in the ongoing Second Extension Study. All of the subjects were in remission at the last date of assessment.

### Example 5

As described in the above Examples, Formulation A has a demonstrated therapeutic action. As described in PCT publication number WO 2009/086634, studies have been conducted to investigate the mechanism of action of Formulation A. In particular, studies were conducted to determine inhibition of binding interactions between radioligands and their receptors, or inhibition of radio-labeled enzymes to act on their associated target proteins by Formulation A. The level of inhibition by Formulation A (measured as percent inhibition of specific binding to each receptor by Formulation A) was determined. The testing of inhibition of binding interactions and enzymatic activities was performed in duplicate for each sample at two different concentrations of Formulation A (1.0 μg/mL and 10.0 μg/mL). These concentrations of Formulation A were prepared by dissolving the contents of a capsule of Formulation A in dimethyl sulfoxide and subsequently diluting the solution to either 1.0 μg/mL or 10.0 μg/mL of Formulation A. These diluted solutions were called Isolate A. Radioligand binding assays were then performed using more than 60 different receptors and enzymes (as described in detail in PCT publication number WO 2009/086634). The average percent inhibition of specific binding at each concentration of Isolate A was then determined.

Of the more than 60 receptors and enzymes tested, 5 receptors showed binding inhibition activity. The results of this study showed that binding by neurokinin A to the human NK₂ receptor was inhibited by 32.15% in the presence of Isolate A (approximately 10 μg/mL). The dissociation constant (K_{d}) was 5×10⁻¹⁰ M and the inhibition constant (Kᵢ) of the reference compound, neurokinin A, was 2.53×10⁻¹⁰ M. In addition, the above-described binding inhibition studies showed that Isolate A displaced glutamate from four of its major ionotropic receptors. Binding by radio-labeled AMPA to the AMPA receptor was inhibited by 29.05% in the presence of Isolate A (approximately 10 μg/mL). Binding by radio-labeled kainic acid to the kainate receptor was inhibited by 22.38% in the presence of Isolate A (approximately 10 μg/mL). Binding by radio-labeled CGP 39653 to the agonist site of the NMDA receptor was inhibited by 34.59% in the presence of Isolate A (approximately 10 μg/mL). Binding by radio-labeled MDL-105,519 to the glycine site that is strychnine-insensitive of the NMDA receptor was inhibited by 27.45% in the presence of Isolate A (approximately 10 μg/mL).

The NK₂ receptor was used in additional receptor binding assays. A one-concentration controlled experiment was performed to assess the ability of various isolates of the contents of a Formulation A capsule to antagonize ligand binding by the NK₂ receptor. The contents of Formulation A capsules were dissolved using various solvents and extracted using four different processes, as described in detail below. These extraction procedures resulted in a number of isolates. These isolates were called: Sample #19 Top Isolate, Sample #19 Bottom Isolate, Sample #20 Top Isolate, Sample #20 Bottom Isolate, Fraction X Isolate and Sample #2 Isolate. These isolates were then each tested in the radioligand binding assay. In order to more easily track the binding activity in the assay, higher concentrations of isolate were used (e.g., approximately 100 μg/ml). This radioligand binding assay was performed based on the methods of Burcher and of Regoli. In general, Chinese Hamster Ovary (CHO) cells expressing recombinant human NK₂ receptor were incubated with [¹²⁵I]neurokinin A (final concentration of 1.0 μM) in the presence of control (neurokinin A) or each of the isolates). The reactions were carried out in 20 mM HEPES (pH 7.4) containing 0.02% bovine serum albumin and 1 mM MnCl₂ for 4 hours at 25° C. The reaction was then terminated by rapid vacuum filtration onto glass fiber filters. Radioactivity trapped onto the filters was measured and compared to control values in order to ascertain any interactions of the isolates with the neurokinin A binding site of the NK₂ receptor (measured as a percentage of specific binding).

Sample #19 was prepared by weighing out 103 mg of the contents of a Formulation A capsule. Water (10.3 mL) was added and the solution was vortexed for one minute. Thirty mL of ethyl acetate was then added to the solution and the solution was vortexed again for 1 minute. The sample was then centrifuged using a bench top Beckman centrifuge. Three fractions were formed as a result. The top (organic) and bottom (aqueous) fractions were collected separately and the middle fraction was discarded. The top and bottom fractions were each dried down. The bottom (aqueous) fraction was reconstituted in 2.06 mL of water. The sample was not clear and it was centrifuged at 10,000 rpm for ten minutes using a microcentrifuge. The supernatant was removed, labeled as sample 085426-4 (Sample #19 Bottom Isolate) and used in the receptor binding studies. The top (organic) fraction was reconstituted in 1.245 mL of 20% acetonitrile in water. The sample was not clear and it was centrifuged at 10,000 rpm for ten minutes using a microcentrifuge. The supernatant was removed, labeled as sample 085426-3 (Sample #19 Top Isolate) and used in the receptor binding studies. A control for sample #19 was also made. This control consisted of 20% acetonitrile in water, and was labeled as sample 085426-5 in the receptor binding studies.

Sample #20 was prepared by weighing out 249.7 mg of the contents of a Formulation A capsule. Ten mL of 1:1 methanol:dichloromethane was added and the solution was vortexed. Ten mL of dichloromethane was then added to the solution and the solution was vortexed again. The sample was then centrifuged at 3500 rpm for fifteen minutes using a bench-top Beckman centrifuge. Three fractions were formed as a result. The top and bottom organic fractions were collected separately. The middle fraction was discarded. The top and bottom fractions were each dried down and reconstituted in 2.49 mL of 100% methanol in water. The top methanol fraction was semi-clear and the bottom dichloromethane fraction was not soluble. Both samples were centrifuged at 10,000 rpm for ten minutes using a microcentrifuge. The supernatant of each sample was removed. The supernatant from the top methanol fraction was labeled as sample 085426-6 (Sample #20 Top Isolate) and used in the receptor binding studies. The supernatant for the bottom dichloromethane fraction was labeled as sample 085426-7 (Sample #20 Bottom Isolate) and used in the receptor binding studies. A control for sample #20 was also made. This control consisted of 10% methanol in water, and was labeled as sample 085426-9 in the receptor binding studies.

Sample Fraction X was prepared as follows. One hundred and twenty-one mg of the contents of a Formulation A capsule was weighed out. Ten mL of water was then added. Ten mL of dichloromethane was then added to the solution and the sample was vortexed. The aqueous and organic fractions were each separately removed. The solvent isolation was repeated by adding 10 mL of dichloromethane to the aqueous fraction and vortexing the solution. Again, the aqueous and organic fractions were each separately removed. The organic fractions from the two isolations were combined and the aqueous fractions from the two isolations were combined. The aqueous and organic fractions were dried down and weighed. The aqueous fraction weighed 116.4 mg and the organic fraction weighed 1.3 mg. The organic fraction was reconstituted in 1.3 mL of 10% methanol in water (for a concentration of 0.1 mg/mL), labeled as sample 085426-8 (Fraction X Isolate) and used in the binding studies. A control for sample Fraction X was also made. This control consisted of 10% methanol in water, and was labeled as sample 085426-9 (note this was the same control as used for sample #20) in the receptor binding studies.

Sample #2 was prepared as follows. A portion (1.8 mg) of the contents of a Formulation A capsule was weighed out. Forty percent PEG in water plus 0.25% Tween 80 (3.6 mL) was then added (for a concentration of 0.5 mg/mL) and the sample was vortexed. This preparation was labeled as sample 085426-1 (Sample #2 Isolate) and was tested in the receptor binding studies. A control for sample #2 was also made. This control consisted of 40% PEG in water plus 0.25% Tween 80, and was labeled as sample 085426-2 in the receptor binding studies.

The results (obtained from duplicate samples of each isolate at maximal concentrations) from the receptor binding study are presented in the following table.

| | # 19 | #19 | | Fraction | #20 | #20 | #20/Fraction | | |
|---|---|---|---|---|---|---|---|---|---|
| Target | Top | Bottom | #19 | X | Top | Bottom | X | #2 | #2 |
| Receptor | Isolate | Isolate | Control | Isolate | Isolate | Isolate | Control | Isolate | Control |
| Neurokinin 2 | 18% | 13% | 12% | 55% | 53% | 18% | 1% | 102% | 102% |

Bolded data indicate more than 50% inhibition at the concentration tested.

The Fraction X Isolate inhibited neurokinin A binding to its NK₂ receptor by 55%, and Sample #20 Top Isolate inhibited neurokinin A binding to its NK₂ receptor by 53%.

To further confirm that binding to and activation of the NK₂ receptor is antagonized by Sample #20 Top Isolate, prepared as described above, dose response studies were conducted. Inhibition of binding of the NK₂ receptor was evaluated in the presence of the following concentrations of #20 Top Isolate (based on the amount of Formulation A contained in Sample #20 prior to extraction): 0.1, 0.3, 1.0, 3.0, 10, 30, 100 and 300 μg/mL.

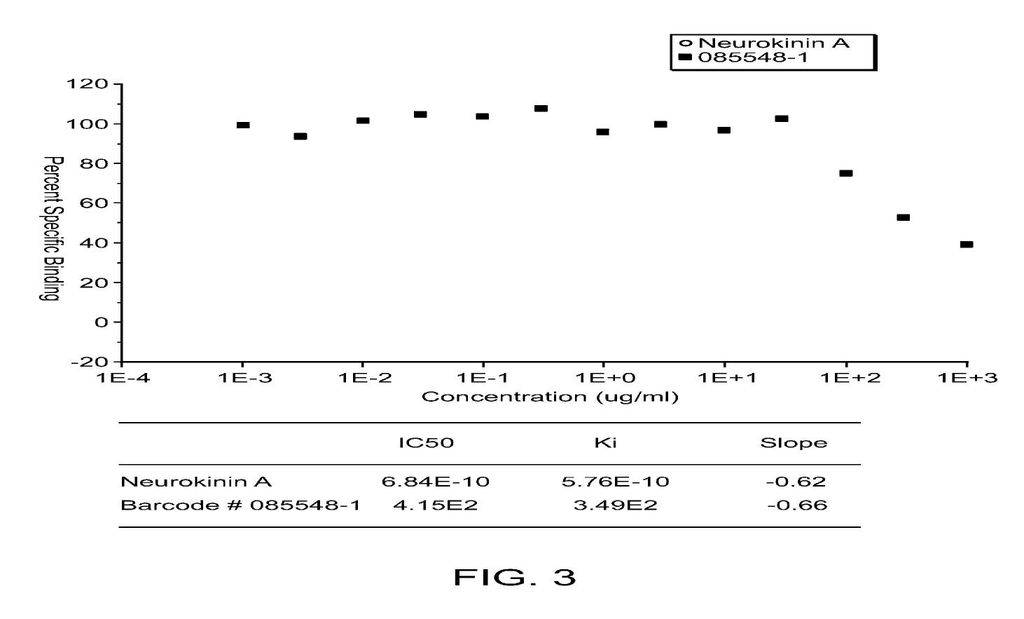

FIG. 3 shows the results of the assay performed with the NK₂ receptor. Sample #20 Top Isolate inhibited the ability of neurokinin A to bind the NK₂ receptor in a concentration-dependent manner, with higher concentrations of Sample #20 Top Isolate providing more binding inhibition and lower concentrations providing less binding inhibition. The IC₅₀ of neurokinin A was determined to be 6.84×10⁻¹⁰ μg/mL and the Kᵢ was determined to be 5.76×10⁻¹⁰ M. The IC₅₀ of Sample #20 Top Isolate was determined to be 4.15×10² μg/mL and the Kᵢ was determined to be 3.49×10² M.

### Example 6

The compound that interacts with the NK_{z} receptor was isolated as follows. A crude extract (1.91 g, off-white amorphous) of Formulation A suspended in water (HPLC Grade, J. T. Baker) was loaded into a WP C18 column (40 μm, J. T. Baker). The packed column was eluted with sequential rounds of water, 30% methanol, 85% methanol and 100% methanol (HPLC solvent, J. T. Baker). Aliquots of these methanol containing fractions were then assayed in the human NK_{z} receptor radioligand binding assay as described above. The results of this assay showed that the greatest inhibition of binding was present in the fractions eluted with 85% methanol and 100% methanol. These two fractions each exhibited 98.2% activity against N₂ when used at a concentration of 0.1 mg/ml. Methanol from methanol containing active fractions (i.e., the fractions eluted with 85% and 100% methanol) was removed under vacuum and the remaining water was removed with lyophilization (Labconco). The dried fractions were stored at −20° C.

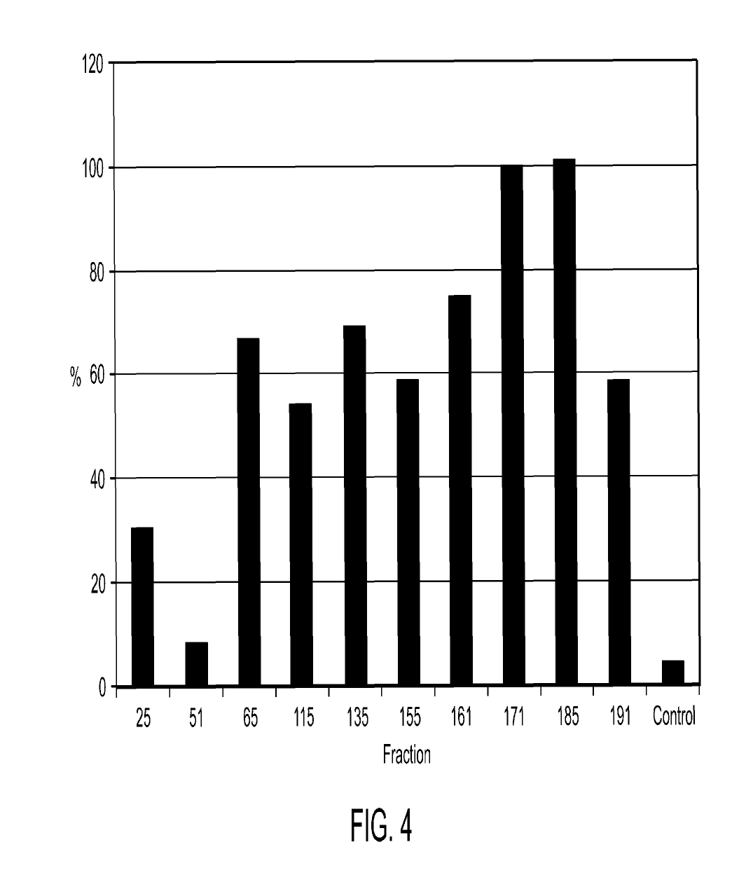

The 0.599 g active fraction (which was a combination of the dried fractions eluted with 85% and 100% methanol), which showed 98.2% activity against the human NK_{z} receptor at 0.1 mg/ml, was applied to a WP C18 column (Φ2.1×50 cm, 40 μm, J. T. Baker). The active components were eluted by applying a multi-step gradient of acetonitrile from 20%, 50% to 70%. (Optima® LC/MS grade, Fisher Scientific). Aliquots of fractions (each fraction containing between 10 and 15 ml of eluate) were again assayed in the human NK_{z} receptor radioligand binding assay as described above. FIG. 4 shows the results of the binding studies for Fractions 25, 51, 65, 115, 135, 155, 161, 171, 185, 191 and control (which contained only eluent (0.05% Methanol). Fractions 171 and 185 showed 99.8% and 100.8% binding inhibition of radio-labeled NKA, respectively. Solvent was removed and dried samples fractions were stored at −20° C.

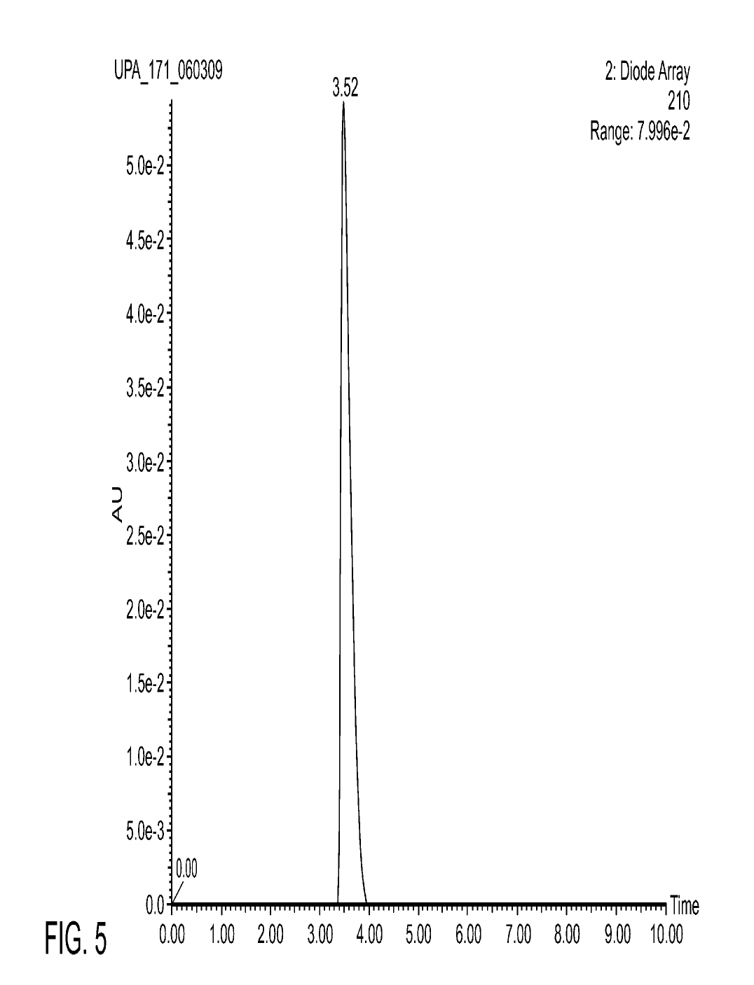

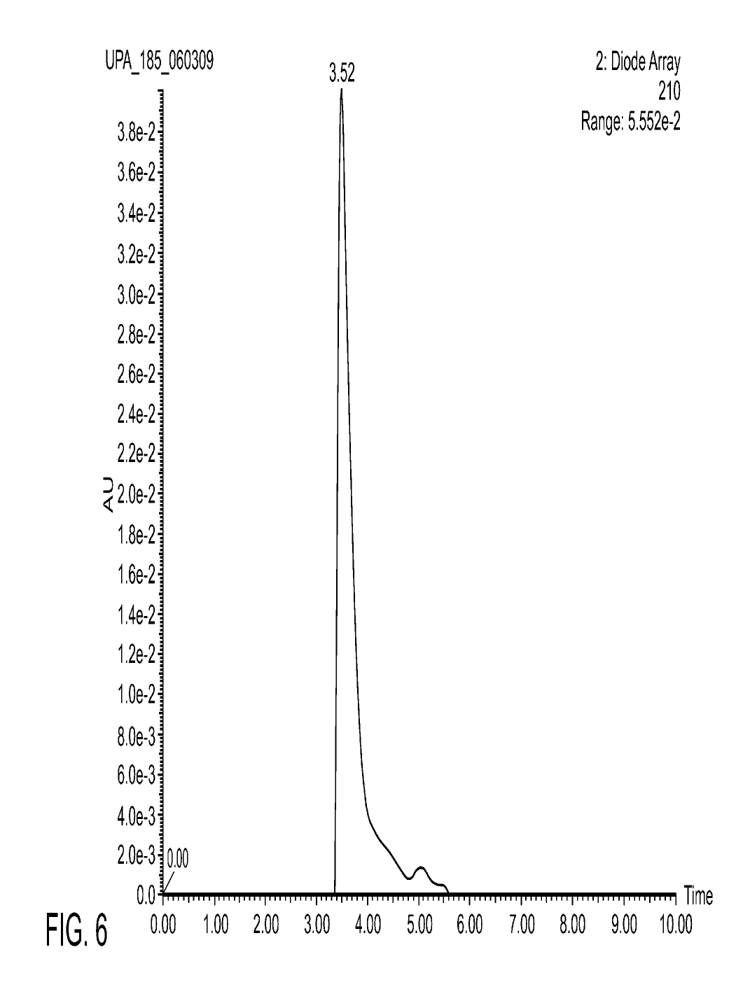

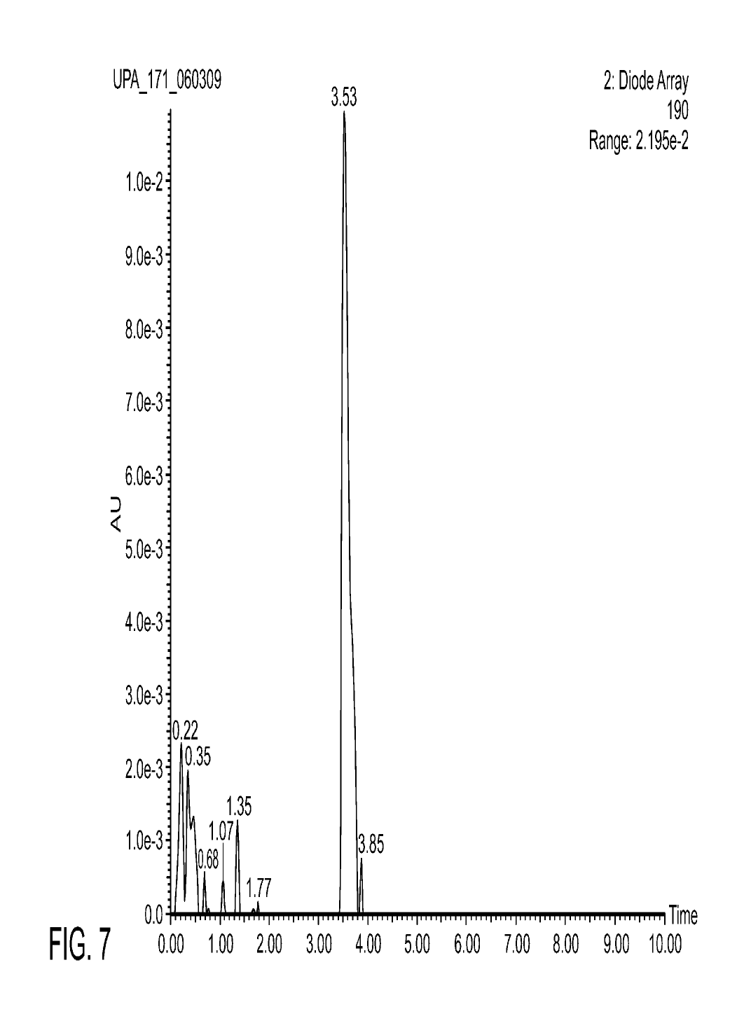

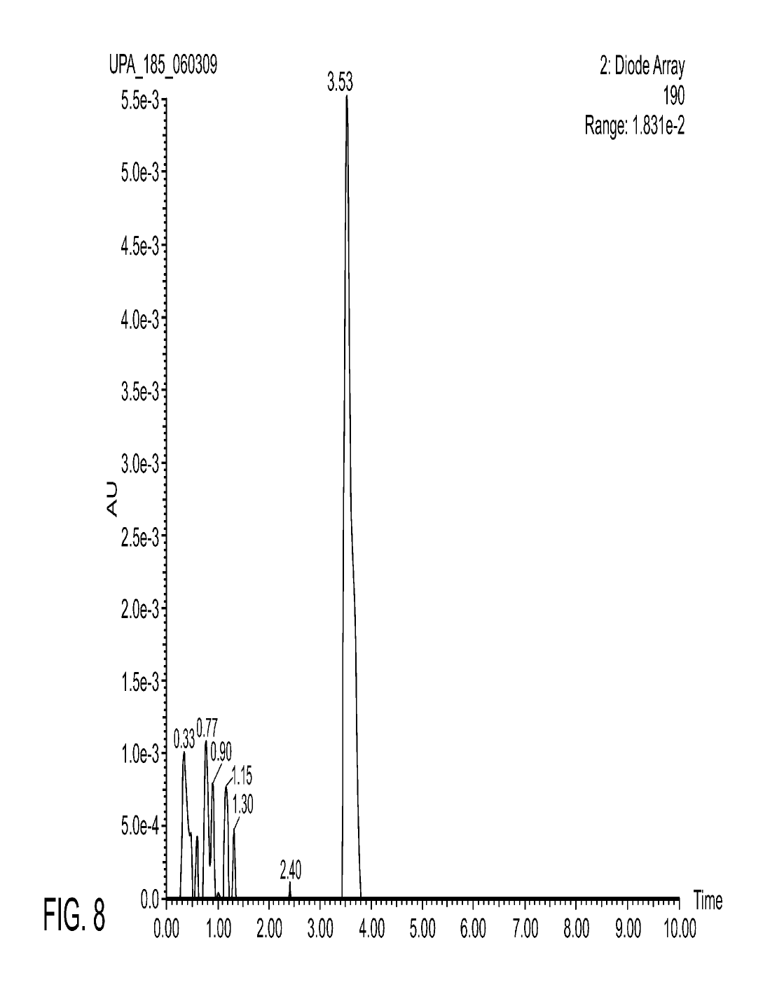

The purity of the most active fractions, from 171 (99.8% against NK₂) and 185 (100.8% against NK₂) was identified using HPLC-UV, using standard conditions known to those skilled in the art. The results of these experiments are shown in FIGS. 5 to 8. FIG. 5 shows a chromatogram of Fraction 171, and FIG. 6 shows a chromatogram shows a chromatogram of Fraction 185, both fractions being detected using 210 nm of light. FIG. 7 shows a chromatogram of Fraction 171, and FIG. 8 shows a chromatogram of Fraction 185, both fractions being detected using 190 nm of light.

A 600 MHz ¹H NMR (Bruker) was used to further assess Fractions 171 and 185. These experiments showed that both fractions were pure and that both fractions contained the same compound. Fractions 170, 171, 172, 173 and 174 were combined into a single sample and assayed using the 600 MHz ¹H NMR HR-mass spectrometry in order to determine the identity of the structure of the compound in the sample (i.e., the compound that binds the NK₂ receptor).

The one-dimensional NMR spectroscopy analysis shows typical ¹H— and ¹³C resonance which accounts for a total of 14 (out of eventual 36) protons and 18 carbons. The NMR assignment are as following for the protons—4.23 (1H, dd, 11.74, 4.70), 4.17 (1H, dd, 11.74, 5.87), 3.95 (1H, m), 3.72 (1H, dd, 11.44, 4.11), 3.62 (1H, dd, 11.44, 5.87), 2.37 (2H, dd, 8.78, 7.62), 1.65 (2H, m), 1.35-1.25 (overlapped), 1.13 (1H, m), 0.88 (3H, overlapped) and 0.86 (3H, overlapped); for ¹³C-174.1 (C=0), 70.2 (OCH), 65.1 (OCH₂), 63.2 (OCH₂), 36.5 (CH₂), 34.3 (CH), 34.0 (CH₂), 29.9 (CH₂), 29.5 (CH₂), 29.5 (CH₂), 29.4 (CH₂), 29.3 (CH₂), 29.1 (CH₂), 29.0 (CH₂), 27.0 (CH₂), 24.8 (CH₂), 19.1 (CH₃) and 11.3 (CH₃). The CH and CH₃ assignments were confirmed using Distortionless Enhancement by Polarization Transfer (DEPT) NMR techniques. The DEPT-90 (middle) and -135 (top) experiments further confirmed that there are two CH₃s and 2CHs. Any positive peaks in DEPT-135 which don't appear in DEPT-90 are CH₃s.

Multidimensional NMR spectroscopy techniques were used to establish the atomic and functional groups connectivity, which lead to the final 2-dimensional structure elucidation.

These studies thus showed that the active compound that binds to the NK₂ receptor has the chemical structure depicted by Formula (I), the stereochemistry of the chiral centers of the isolated compound being unassigned.

First, the heteronuclear single-quantum correlation (HSQC, H-C) further confirmed the assignment of the units of CH, CH₂ and CH₃ from the one-dimensional experiment.

Double-quantum filtered (DQFCOSY) and total correlation (TOCSY, or Homonuclear Hartmann Hahn, HOHAHA) experiments established the back-bone connectivities of the glyceride unit. The DQFCOSY measures the connectivities of adjacent protons, whereas the TOSCY may reach through several bonds.

The longer range connectivities (beyond glyceride) were establish using heteronuclear correlation techniques, namely, HMBC (heteronuclear multiple-bond correlation). This method allowed for the connection of the glycerol (C1-H) to the myristic carbonyl through the carboxyl (ester) linkages. It also provided the linkage between the C-alpha and -beta protons with the carbonyl of the carboxyl functionality. Connectivity of the C-alpha and -beta protons could also be extended to include some the CH₂ groups of the myristic acid adjacent to them.

At the conclusion of the NMR analysis, the compound that bound the NK₂ receptor was identified as the compound of Formula (I). This assignment resulted in the proposed molecular formula as C₁₈H₃₆O₄ with the corresponding molecular weigh of approx. 316, which was then confirmed by AccuTOF experiments.

### Example 7

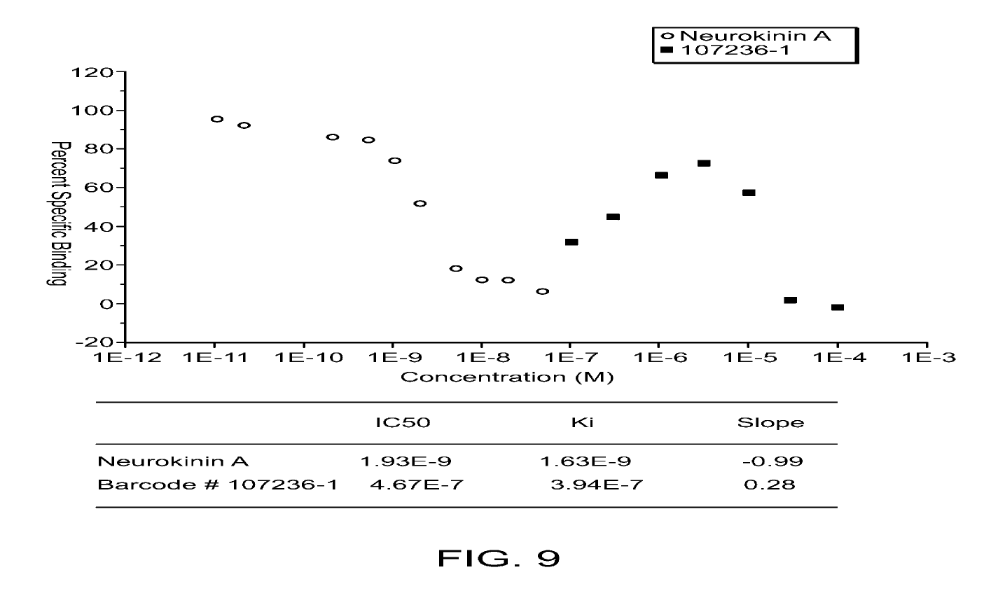

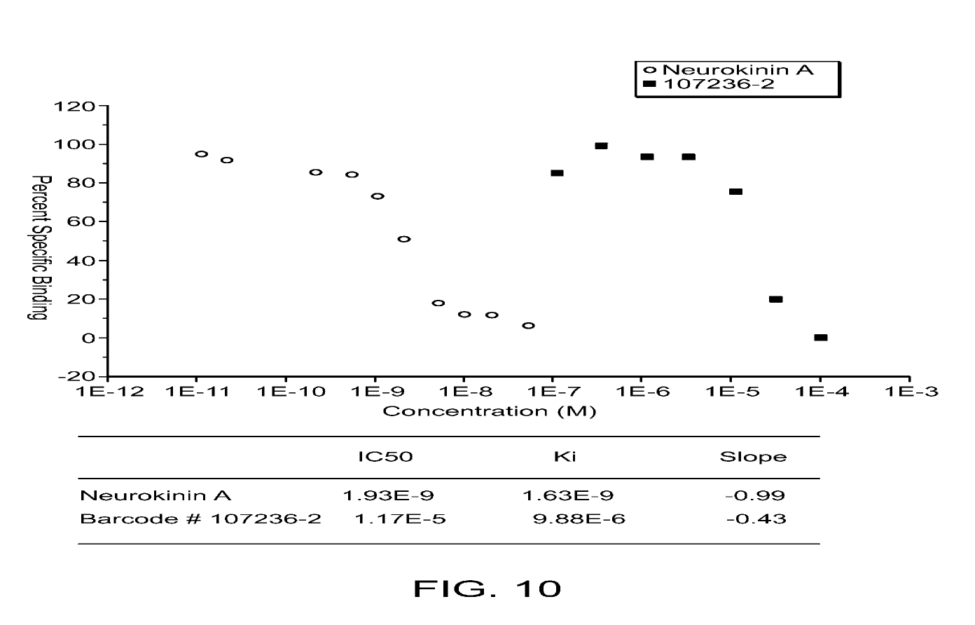

6-methyl-myristic acid 2,3-dihydroxypropyl ester was synthesized as described above, and myristic acid 2,3-dihydroxypropyl ester was obtained, and the compounds dissolved in dimethyl sulfoxide (DMSO). The effects of various concentrations of 6-methyl-myristic acid 2,3-dihydroxypropyl ester and myristic acid 2,3-dihydroxypropyl ester on the human NK₂ receptor binding assay described above in Example 5 were tested. FIG. 9 shows the results of the assay performed with the NK₂ receptor in the presence of 6-methyl-myristic acid 2,3-dihydroxypropyl ester (labeled in FIG. 10 as compound “107236-1”). In general, 6-methyl-myristic acid 2,3-dihydroxypropyl ester inhibited the ability of neurokinin A to bind the NK₂ receptor in a concentration-dependent manner. The IC₅₀ Kᵢ of neurokinin A and 6-methyl-myristic acid 2,3-dihydroxypropyl ester are shown in FIG. 9.

FIG. 10 shows the results of the radioligand binding assay performed with the NK₂ receptor in the presence of myristic acid 2,3-dihydroxypropyl ester (labeled in FIG. 10 as compound “107236-2”). Myristic acid 2,3-dihydroxypropyl ester also inhibited the ability of neurokinin A to bind the NK₂ receptor. The IC₅₀ Kᵢ of neurokinin A and myristic acid 2,3-dihydroxypropyl ester are shown in FIG. 10.

### Example 8

6-methyl-myristic acid 2,3-dihydroxypropyl ester was synthesized as described above, and myristic acid 2,3-dihydroxypropyl ester was obtained. They were each prepared as solutions by addition of dimethyl sulfoxide (DMSO) to a concentration of 10 mM. These compounds were then used in cellular/functional calcium flux agonist and antagonist assays to determine the effect the compounds have on human NK₂ receptor activity as measured by changes intracellular calcium measurements. These assays were carried out based on the method of Gerard et al.

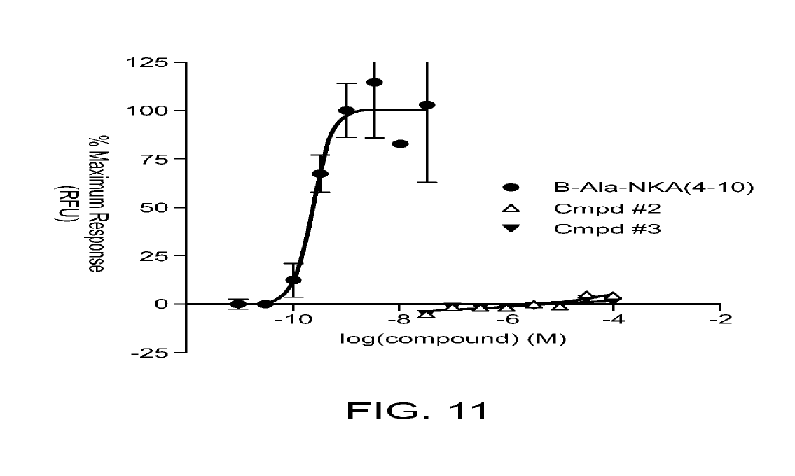

Briefly, the NK₂ receptor agonist assay was performed as follows. Chinese Hampster Ovary-K1 (CHO—K1) cells stably expressing recombinant human NK₂ receptor were plated on mixed extracellular matrix overnight in complete media. One hour before the assay, the media was replaced with Hank's Buffered Salt Solution (HBSS) containing 0.1% bovine serum albumin. The cells were then loaded with dye that measures intracellular calcium and baseline measurements of intracellular calcium were taken. Control (agonist [bAla⁸]-NKA (4-10) at concentrations ranging from 1×10⁻¹¹ M to 3×10⁻⁷ M) or compound (myristic acid 2,3-dihydroxypropyl ester (compound 2) or 6-methyl-myristic acid 2,3-dihydroxypropyl ester (compound 3), each at concentrations ranging from 3×10⁻⁷ M to 1×10⁻⁴ M), were then added to the appropriate wells of cells. Fluorescence at 485 nm excitation/515 nm emission was measured every 2 seconds for at least two minutes. The peak height of fluorescence in each of the wells receiving compound 2 or compound 3 was recorded and compared to the peak height of fluorescence in the wells receiving the control. The results of this assay are presented in FIG. 11, which is a graph of the percent maximum value for the [bAla⁸]-NKA (4-10) control (% Maximum Response) versus the concentration of compound for the control, myristic acid 2,3-dihydroxypropyl ester (cmpd #2) or 6-methyl-myristic acid 2,3-dihydroxypropyl ester (cmpd #3) (log(compound) (M)).

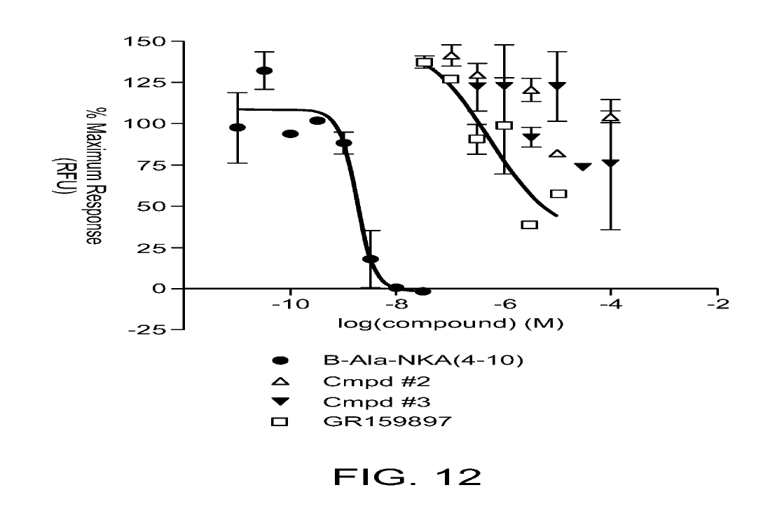

The NK₂ receptor antagonist assay was performed essentially as follows. CHO-K1 cells stably expressing recombinant human NK₂ receptor were plated on mixed extracellular matrix overnight in complete media. One hour before the assay, the media was replaced with Hank's Buffered Salt Solution (HBSS) containing 0.1% bovine serum albumin. The cells were then loaded with dye that measures intracellular calcium and baseline measurements of intracellular calcium were taken. Controls (antagonist GR 159897 at concentrations ranging from 3×10⁻⁸ M to 1×10⁻⁵ M or [bAla⁸]-NKA (4-10) at concentrations ranging from 1×10⁻¹¹ M to 3×10⁻⁷ M) (the calcium effects of [bAla⁸]-NKA (4-10) dissipate over time, therefore, [bAla⁸]-NKA (4-10) can act like an antagonist in the antagonist assay, by blocking additional effects of [bAla⁸]-NKA (4-10)) or sample (myristic acid 2,3-dihydroxypropyl ester (compound 2) or 6-methyl-myristic acid 2,3-dihydroxypropyl ester (compound 3), each at concentrations ranging from 3×10⁻⁷ M to 1×10⁻⁴ M, were then added to the appropriate wells of cells. After ten minutes, agonist [bAla]-NKA (4-10) (0.3 nM final concentration) was added. Fluorescence at 485 nm excitation/515 nm emission was measured every 2 seconds for at least two minutes. The peak height of fluorescence in each of the wells receiving controls, compound 2 or compound 3 was recorded and compared to the peak height of fluorescence in the wells receiving only the agonist. The results of this assay are presented in FIG. 12, which is a graph of the percent maximum value for the [bAla⁸]-NKA (4-10) control (% Maximum Response) versus the concentration of compound for the controls, myristic acid 2,3-dihydroxypropyl ester (cmpd #2) or 6-methyl-myristic acid 2,3-dihydroxypropyl ester (cmpd #3) (log(compound) (M)). As shown in FIG. 12, each of myristic acid 2,3-dihydroxypropyl ester and 6-methyl-myristic acid 2,3-dihydroxypropyl ester displayed antagonist activity in this functional NK₂ receptor antagonist assay.

It is believed that the preparation and use of compounds of the present invention, including individual compounds encompassed by Formula 1, will be apparent from the foregoing description of exemplary embodiments, and may thus be claimed as such. It will be obvious to a person of ordinary skill in the art that various changes and modifications may be made herein without departing from the spirit and the scope of the invention.

### REFERENCES

Ahlstedt I, Engberg S, Smith J, Perey C, Moody A, Morten J, Lagerström-Fermér M, Drmota T, von Mentzer B, Påhlman I, Lindström E, Occurrence and pharmacological characterization of four human tachykinin NK_{z} receptors. Biochemical Pharmacology 2008 76:476-481.
Bai, T, Zhou D, Weir T, Hegele R, Hayashi S, McKay K, Bondy G, Fong T, Substance P (NK₁)- and neurokinin A (NK₂)-receptor gene expression in inflammatory airway diseases. Am J Physiol Lung Cell Mol Physiol 1995 269:L309-L317.
Beck A T, Ward C H, Mock J, Erbaugh J, An inventory for measuring depression. Archives of General Psychiatry 4:561-571, 1961.
Burcher E, Buck S H, Lovenberg W, Characterization and autoradiographic localization of multiple tachykinin binding sites in gastrointestinal tract and bladder. Jrnl. Pharmac. & Exp. Ther. 1986 236(3):819-831.
Dableh L J, Yashpal K, Rochford J, Henry J L, Antidepressant-like effects of neurokinin receptor antagonists in the forced swim test in the rat. Eur J. Pharmacol. 2005 Jan. 10; 507(1-3):99-105. Epub 2004 Dec. 28.
Evangelista S, Involvement of tachykinins in intestinal inflammation. Curr Pharm Des 2001 January; 7(1):19-30.
Gerard N P, Eddy R L Jr, Shows T B, Gerard C, The human neurokinin A (substance K) receptor. Molecular cloning of the gene, chromosome localization, and isolation of cDNA from tracheal and gastric tissues. J Biol Chem 1990 265:20455-20462.
Griebel G, Perrault G, Soubrié P, Effects of SR48968, a selective non-peptide NK₂ receptor antagonist on emotional processes in rodents. Psychopharmacology (Berl). 2001 November; 158(3):241-251.
Guy W: ECDEU Assessment Manual for Psychopharmacology—Revised (DHEW Publ No ADM 76-338). Rockville, Md., U.S. Department of Health, Education, and Welfare, Public Health Service, Alcohol, Drug Abuse, and Mental Health Administration, NIMH Psychopharmacology Research Branch, Division of Extramural Research Programs, 1976, pp 218-222.
Hamilton, Development of a rating scale for primary depressive illness. Br J Soc Clin Psychiatry 1967; 6:278-279.
Hamilton M, The assessment of anxiety states by rating. Br J Med Psychol 1959; 32:50-55.
Holmes A, Heilig M, Rupniak N M, Steckler T, Griebel G, Neuropeptide systems as novel therapeutic targets for depression and anxiety disorders. Trends Pharmacol Sci. 2003 November; 24(11):580-8.
Husum H, Wörtwein G, Andersson W, Bolwig T G, Mathé A A, Gene-environment interaction affects substance P and neurokinin A in the entorhinal cortex and periaqueductal grey in a genetic animal model of depression: implications for the pathophysiology of depression. Int J Neuropsychopharmacol. 2008 February; 11(1):93-101. Epub 2007 May 4.
Khawaja A M, Rogers D F, Tachykinins receptor to effector. Int J Biochem Cell Biol. 1996; 28(7): 721-738.
Kendler K S, Walters E E, Kessler R C, The prediction of length of major depressive episodes: results from an epidemiological sample of female twins. Psychol Med 1997; 27: 107-117.
Lecci A, Capriati A, Maggi C A, Tachykinin NK₂ receptor antagonists for the treatment of irritable bowel syndrome. Br J. Pharmacol. 2004 April; 141(8):1249-63. Epub 2004 Mar. 22.
Lingjaerde I, Ahlfors U G, Bech P, et al, The UKU Side Effect Rating Scale: a new comprehensive rating scale for psychotropic drugs, and a cross-sectional study of side effects in neuroleptic-treated patients. Acta Psychiatrica Scandinavica Suppl 76:1-100, 1987.
Louis C, Stemmelin J, Boulay D, Bergis O, Cohen C, Griebel G, Additional evidence for anxiolytic- and antidepressant-like activities of saredutant (SR48968), an antagonist at the neurokinin-2 receptor in various rodent-models. Pharmacol Biochem Behay. 2008 March; 89(1):36-45. Epub 2007 Nov. 5.
McLeod J D, Kessler R C, Landis K R, Recovery from major depressive episodes in a community sample of married men and women. J Abnorm Psychol 1992; 101: 277-286.
Micale V, Tamburella A, Leggio G M, Mazzola C, Li Volsi V, Drago F, Behavioral effects of saredutant, a tachykinin NK₂ receptor antagonist, in experimental models of mood disorders under basal and stress-related conditions. Pharmacol Biochem Behay. 2008 September; 90(3):463-9. Epub 2008 Apr. 12.
Montgomery S A, Asberg M, A new depression scale designed to be sensitive to change. British Journal of Psychiatry 134:382-389, 1979.
Patten S, The duration of major depressive disorders in the Canadian general population. Chronic Dis Canada 22:1.2001.
Pinto F M, Almeida T A, Hermandez M, Devillier P, Advenier C, Luz Candenas M, mRNA expression of tachykinins and tachykinin receptors in different human tissues. European Journal of Pharmacology 2004 494:233-239.
Regoli D and Nantel F, Pharmacology of neurokinin receptors, Biopolymers. 1991 31:777-783.
Rizzo C A, Hey J A, Activity of nonpeptide tachykinin antagonists on neurokinin a induced contractions in dog urinary bladder. J Urol. 2000 June; 163(6):1971-4.
SaloméN, Stemmelin J, Cohen C, Griebel G, Selective blockade of NK₂ or NK₃ receptors produces anxiolytic- and antidepressant-like effects in gerbils. Pharmacol Biochem Behay. 2006 April; 83(4):533-9. Epub 2006 Apr. 19.
Steinberg R, Alonso R, Griebel G, Bert L, Jung M, Oury-Donat F, Poncelet M, Gueudet C, Desvignes C, Le Fur G, Soubrié P, Selective blockade of neurokinin-2 receptors produces antidepressant-like effects associated with reduced corticotropin-releasing factor function. J Pharmacol Exp Ther. 2001 November; 299(2):449-58.
Stratton S C, Beresford I J, Harvey F J, Turpin M P, Hagan R M, Tyers M B, Anxiolytic activity of tachykinin NK₂ receptor antagonists in the mouse light-dark box. Eur J. Pharmacol. 1993 Dec. 21; 250(3):R11-2.
Teixeira R M, Santos A R, Ribeiro S J, Calixto J B, Rae G A, De Lima T C, Effects of central administration of tachykinin receptor agonists and antagonists on plus-maze behavior in mice. Eur J. Pharmacol. 1996 Sep. 5; 311(1):7-14.
Toulouse M, Coelho A, Fioramonti J, Lecci A, Maggi C, Bueno L, Role of tachykinin NK₂ receptors in normal and altered rectal sensitivity in rats. Br J Pharmacology 2000 129, 193-199.
Treatment of Chronic Depression (Editorial), NEJM 342:1518-1520, 2000.
Walsh D M, Stratton S C, Harvey F J, Beresford I J, Hagan R M, The anxiolytic-like activity of GR159897, a non-peptide NK₂ receptor antagonist, in rodent and primate models of anxiety. Psychopharmacology (Berl). 1995 September; 121(2):186-91.
Ware J E Jr, Sherbourne C D, The MOS 36-item short-form health survey (SF-36). 1. Conceptual framework and item selection Medical Care 1992, 30:473-483.

1-76. (canceled)

77. A compound having the following the structure: 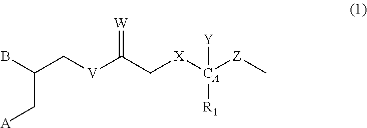
wherein:
A and B are independently —OH or —SH,
V and W are independently oxygen or sulfur and at least one of V and W is oxygen,
R₁ is —(CH₂)ₚCH₃ or is —H, and
p is an integer from 0 to 3, and:
X is —(CH₂)ₘ—,
Y is —H,
Z is —(CH₂)ₙ—,
m and n are integers,
m=1 to 5,
n=4 to 14,
6≦m+n≦14 for all m and n, and
wherein, optionally, there are up to two carbon-carbon double bonds, each double bond formed between adjacent methylene groups of formula (1) wherein, if there are two said double bonds each carbon thereof is bonded to at least one hydrogen;
or X is 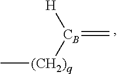
Y is absent, and C_{A} and C_{B} together form a double bond,
Z is —(CH₂)ᵣ—,
q and r are integers,
q=0 to 4,
r=1 to 13,
5≦q+r≦13 for all q and 4, and
wherein, optionally, there is a second double bond formed between adjacent methylene groups of formula (1) wherein each carbon thereof is bonded to at least one hydrogen;
or X is —(CH₂)ₜ—,
Z is 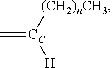
Y is absent, and C_{A} and C_{C} together form a double bond,
R₁ is —(CH₂)ᵥCH₃ or is —H,
t and u are integers,
t=1 to 5,
u=0 to 12,
5≦t+u≦13 for all t and u, and
wherein, optionally, there is a second double bond formed between adjacent methylene groups of formula (1) wherein each carbon thereof is bonded to at least one hydrogen,
including or a pharmaceutically acceptable salt of the compound.

78. The compound of claim 77, wherein A and B are both —OH.

79. The compound of claim 77, wherein V and W are both oxygen.

80. The compound of claim 77, wherein R₁ is —(CH₂)ₚCH₃.

81. The compound of claim 77, wherein p is 0 to 2.

82. The compound of claim 77, wherein p is 0 or 1.

83. The compound of claim 77, wherein p is 0.

84. The compound of claim 77, wherein n=2 to 12 and 7≦m+n≦13.

85. The compound of claim 77, wherein n=3 to 11 and 8≦m+n≦12.

86. The compound of claim 77, wherein n=4 to 10 and 9≦m+n≦11.

87. The compound of claim 77, wherein n=5 to 9 and m+n=10.

88. The compound of claim 77, wherein m=2 to 4.

89. The compound of claim 77, wherein m=3.

90. The compound of claim 77, wherein r=2 to 12 and 6≦q+r≦12.

91. The compound of claim 77, wherein r=3 to 11 and 7≦q+r≦11.

92. The compound of claim 77, wherein r=4 to 10 and 8≦q+r≦10.

93. The compound of claim 77, wherein r=5 to 9 and q+r=9.

94. The compound of claim 77, wherein q=1 to 3.

95. The compound of claim 77, wherein q=2.

96. The compound of claim 77, wherein u=1 to 11 and 6≦t+u≦12.

97. The compound of claim 77, wherein u=2 to 10 and 7≦t+u≦11.

98. The compound of claim 77, wherein u=3 to 9 and 8≦t+u≦10.

99. The compound of claim 77, wherein u=4 to 8 and t+u=9.

100. The compound of claim 77, wherein t=2 to 4.

101. The compound of claim 77, wherein t=3.

102. The compound of claim 77, wherein if said up to two carbon-carbon double bonds are present, then each said bond is formed between methylene groups of Z.

103. The compound of claim 102, wherein said up to two carbon-carbon double bonds is a one said bond.

104. The compound of claim 77, wherein if said second double bond is present, then said bond is formed between methylene groups of Z.

105. The compound of claim 77, wherein said up to two carbon-carbon double bonds and said second double bond are absent.

106. A compound of claim 77 having the following formula:

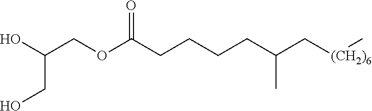

107. A substantially stereochemically pure compound of claim 77 having the formula:

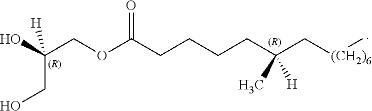

108. A substantially stereochemically pure compound of claim 77 having the formula:

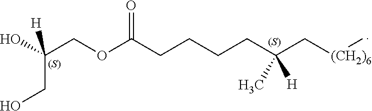

109. A substantially stereochemically pure compound of claim 77 having the formula:

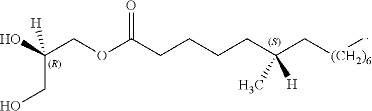

110. A substantially stereochemically pure compound of claim 77 having the formula:

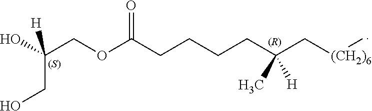

111. A pharmaceutical composition comprising the compound of claim 77 and a pharmaceutically acceptable carrier.

112. A dosage form comprising the compound of claim 77.

113. A method for treating a disorder or disease associated with neurokinin 2 (NK₂) receptor activity, said method comprising the step of administering a therapeutically effective amount of the compound, or a pharmaceutically acceptable salt thereof, of claim 77 to a subject in need thereof.

114. The method of claim 77, in which A and B are each —OH, V and W are oxygen, R₁ is H, X is —(CH₂)ₘ—, Z is —(CH₂)ₙ—, m is 3, n is 7, and the compound contains no C═C double bonds, to a subject in need thereof.

115. The method of claim 114, wherein said disorder or disease associated with said NK₂ receptor activity is a depressive mood disorder, anxiety disorder, irritable bowel syndrome, inflammatory bowel disease, inflammatory airway disease or urinary incontinence.

116. The method of claim 113, wherein the compound is contained in a pharmaceutical formulation comprising a pharmaceutically acceptable carrier.

117. A method for treating a disorder or syndrome associated with a depressive mood disorder, said method comprising the step of administering a therapeutically effective amount of the compound, or pharmaceutically acceptable salt thereof, of claim 77 to a subject in need thereof.

118. The method of claim 117, in which A and B are each —OH, V and W are oxygen, R₁ is H, X is —(CH₂)ₘ—, Z is —(CH₂)ₙ—, m is 3, n is 7, and the compound contains no C═C double bonds, to a subject in need thereof.

119. The method of claim 117, wherein the disorder or syndrome is a disorder of the brain or nervous system, anxiety disorder, sexual dysfunction, substance abuse, eating disorder or hormone disorder.

120. A method of treating a disorder or condition treatable by an antidepressant, said method comprising the step of administering a therapeutically effective amount of the compound, or pharmaceutically acceptable salt thereof, of claim 77 to a subject in need thereof.

121. The method of claim 120, in which A and B are each —OH, V and W are oxygen, R₁ is H, X is —(CH₂)ₘ—, Z is —(CH₂)ₙ—, m is 3, n is 7, and the compound contains no C═C double bonds, to a subject in need thereof.

122. A method for modulating an activity of an NK₂ receptor comprising contacting the NK₂ receptor with an effective amount of the compound, or a pharmaceutically acceptable salt thereof, of claim 77.

123. The method of claim 122, in which A and B are each —OH, V and W are oxygen, R₁ is H, X is —(CH₂)ₘ—, Z is —(CH₂)ₙ—, m is 3, n is 7, and the compound contains no C═C double bonds.
